(19) United States
(12) Patent Application Publication
(10) Pub. No.: US 2026/0000491 A1
(43) Pub. Date: Jan. 1, 2026

(54) Flanged Implant Abutment

(71) Applicant: Eric Knutson, Granite Bay, CA (US)

(72) Inventor: Eric Knutson, Granite Bay, CA (US)

(21) Appl. No.: 18/759,826

(22) Filed: Jun. 29, 2024

(51) Int. Cl. A61C 8/00; A61F 2/28

(52) U.S. Cl. CPC A61C 8/008; A61F 2/2846

(57) ABSTRACT

A flanged healing abutment for securing to an implant that has a bone graft. The flexible flange extends out laterally from the healing abutment body to contact the adjacent gingiva. The flange securely contacts the gingiva in a manner that contains the bone graft, and prevents bacterial infection of the bone graft. The flanged abutment is also able to form gingival emergence profiles. A method for securing a prosthesis to an implant utilizing a flanged healing abutment is also provided.

### FIELD OF INVENTION

The invention relates to dental implant healing abutments, implant bone graft containment, and implant gingival emergence profiles.

### BACKGROUND

After a failing tooth is removed from a site within an osseous structure, such as a jaw, an open osseous and gingival void having a given shape and volume remains open to the oral cavity. The void shape and volume are determined by the shape and volume of the tooth that occupied the site, by bone lost from inflammatory resorption processes, and by bone lost during surgical procedures.

A goal is to restore the osseous and gingival voids to a condition that is similar to the condition prior to the loss of the tooth. To accomplish that, an implant and bone graft are placed into the osseous void, and protected while the bone graft stabilizes. The gingiva is restored by providing a healing abutment to act as a template that forms the healing gingiva to an ideal emergence profile. After a healing period, a prosthesis is connected to the implant to replace the missing tooth.

With rare exceptions, healing abutments are comprised of symmetrical monolithic titanium. Titanium provides a clean hard smooth surface that does not harbor bacteria, is hypoallergenic, has structural simplicity, engages drive tools for securing into implants, and will not cause galvanism. A few healing abutments are comprised of a symmetrical zirconia body connected to a central titanium screw. The symmetry of such hard healing abutments permits rotational seating into implants without excessive interferences with adjacent hard structures.

The diameter of hard healing abutments is constricted, also to avoid excessive interferences with adjacent hard structures. As such the diameter of hard healing abutments is typically less than that of the original tooth being replaced in at least one dimension. Because the diameters of healing abutments are typically smaller than the original tooth in at least one dimension, an undesirable open and uncovered area tends to remain between the adjacent gingiva and healing abutment.

The adjacent gingiva has a given gingival perimeter that constitutes the surface of the gingiva that can be considered to be generally perpendicular to the underlying bone. The gingival perimeter entirely surrounds the implant and bone graft. The configuration of the gingival perimeter is variable depending on the procedural stages. The gingival perimeter configuration fits closely against a tooth when a tooth is present. After a healing period of time, the gingival perimeter configuration changes to fit closely against a healing abutment, and thereby form an emergence profile configuration.

At the initial implant and bone graft placement, portions of the healing abutment perimeter typically are in close contact with the adjacent gingival perimeter. Such close contact between the healing abutment and the gingival perimeter constitutes an adequate biological seal to seal out bacteria from intruding into a bone graft, called a bioseal. The gingiva and ambient blood can rapidly form a bioseal when the gingiva is fitted to closely adapt against the healing abutment.

After a healing period, the gingiva grows in toward the healing abutment perimeter so that the gingiva comes into close contact with the entire perimeter of the healing abutment. The healed gingival perimeter configuration as shaped by the healing abutment constitutes the gingival emergence profile. The gingival emergence profile will largely determine the possible configurations of a prosthesis fabricated to connect to the implant.

When an implant is placed into a portion of the osseous void, the implant also typically has a diameter that is significantly smaller than that of the original tooth within the osseous void, and smaller than the diameter of the gingival perimeter. It is advantageous to occlude the remaining volume of the open osseous void with bone grafts to improve bony support of the implant during and after healing, and to minimize the loss of surrounding alveolar bone volume. The bone graft is substantially contained by the osseous void on all sides, except for the side that faces the oral cavity, which is initially exposed to the oral cavity.

Recently placed bone grafts typically lack cohesive stability, and therefore the bone graft particulates tend to become inadvertently dislodged and lost from the exposed surface into the oral cavity. Exposed bone grafts are also susceptible to invasion and infection from oral bacteria. Such infection can result in failure of the bone graft, failure of the implant, and place the patient's overall health at a degree of risk.

Besides controlling gingival healing, the healing abutment at least partly covers and contains bone grafts. Healing abutments are connected to the implant platform. The portion of a healing abutments that protrudes above the implant platform can be called the body of the healing abutment. The body is considered to have a given height and width, typically assigned to be the greatest vertical and lateral dimensions thereof. The healing abutment typically has a diameter that is greater than that of the implant, and therefore at least partly covers the bone graft, and may even contact a portion of the gingival perimeter.

Often the gingival perimeter is approximated against at least a portion of the healing abutment body. The bone graft that is covered by the contact between the gingiva and the implant body is considered sufficiently contained to resist loss of bone grafts, and to form a biological seal for resisting bacterial intrusion and infection. The interface of the gingiva and the healing abutment body is able to contain the bone graft, and bioseals the bone graft.

It is frequently impractical to approximate the gingiva against the healing abutment to form a gingiva-healing abutment interface around the entire perimeter of the healing abutment body. This is primarily because the healing abutment body occupies a much smaller lateral space over the osseous void than the tooth formerly occupied.

A larger rigid symmetrical healing abutment that has a sufficiently large lateral dimension, or width, to contact the perimeter gingiva typically could not be rotationally secured into the implant platform. This is because the perimeter of a wide healing abutment would typically interfere with adjacent hard structures, and such interference would prevent the rotations needed for rotational securing into the implant.

In rare cases where no adjacent hard structures are present to create an interference, then portions of the larger rigid healing abutment would excessively overhang the gingival perimeter, thereby creating a bacterial and debris trap around those portions of the gingival perimeter. The excessive overhang would also exaggerate the incidence of inadvertent oral forces impacting the hard healing abutment, and thence be transferred to the integrating implant. Excessive overhangs also tend to interfere with normal oral functioning, and be obtrusive to patients.

The greatest lateral diameter of the osseous void is typically much larger than the diameter of the implant. Also, implants are typically placed away from the center of the osseous void, and are therefore asymmetrically positioned within the osseous void. As such, the surface of a bone graft placed in the osseous void has a much larger greatest diameter than the implant diameter. The bone graft therefore extends a given distance laterally from the implant. As such, a connected healing abutment will be closest to the gingival perimeter on one side of the implant, and furthest from the gingival perimeter on another side.

The bone graft is therefore uncovered over a given lateral distance from the implant, and from a healing abutment connected to the implant. A hard healing abutment cannot readily be reconfigured in order to sealingly contact the gingival perimeter, or to cover the bone graft.

The remaining uncovered portions of the bone graft are typically contained by materials that are placed over the surface of the bone graft. The most commonly used type of bone graft covers are membranes. Membranes are typically left in place for several weeks until the bone graft achieves an initial stabilization, such that the bone graft is then able to resist dislodgement and infection. Available membranes covers include non-absorbable PTFE membranes, absorbable collagen membranes, absorbable collagen with bone particles, and so on.

Membranes are not sealingly connected to the healing abutment, and are not integral with the healing abutment. Most membranes are manually trimmed so that the perimeter approximately abuts closely against the perimeter of the healing abutment, with small gaps remaining. Ambient blood can help to bioseal the smaller gaps, but some gaps are of sufficient size so that bacteria are therefore not adequately sealed out from intruding into the bone graft. Such unpredictable biosealing can account for some implant failures.

Membranes have been placed wherein a hole is made in the cover, and the healing abutment is placed through the hole when securing to the implant platform. While the hole connection provides a degree of location stabilization, it is not a biosealing connection, and permits bacterial intrusion through the membrane-healing abutment interface.

At the membrane-gingiva interface, membranes are typically stabilized by laboriously tucking the outer membrane perimeter under surgically formed facial/buccal and lingual/palatal pouches between the gingiva and the surrounding alveolar bone, and tediously stitching to secure the position. Bone graft covers need to remain undisturbed for several weeks until the bone graft becomes dimensionally and biologically stable.

Graft covers and membranes of the art have disadvantages. For example, even after tedious and skillful bone graft cover placement, covers and membranes can inadvertently and prematurely become partly or completely displaced. Graft cover displacement can cause bone graft particulates to become lost from the surface, and can permit bacterial intrusion and infection with the potential of implant loss. As another example, absorbable membranes can absorb prior to bone graft stabilization, and put the bone graft and implant at risk of infection and failure.

After membranes and covers have been in place for a few weeks, the ability to bioseal the bone graft site becomes compromised by gaps that open along the membrane-gingiva interface at the gingival perimeter. Gaps comprise portions of the membrane outer perimeter that do not biosealingly contact the perimeter gingiva, and constitute a route for bacterial intrusion. Gaps tend to form due to dislodgement of the membrane of cover due to inadvertent oral forces, and due to changes in the configuration of the healing perimeter gingiva. Over time, the tendency to form gaps increases rapidly.

When bacteria intrude under a membrane, the membrane constitutes a protective cover for the bacteria, and facilitates rapid bacterial colonization of the site. When gaps are detected, the membrane or cover must therefore be removed immediately, even if it is premature. When membranes are removed prior to adequate bone graft stabilization, then the bone graft has a degree of risk of losing graft particulates or becoming infected. Once the bone graft heals to have adequate stabilization, the membrane is removed to prevent gingival inflammation from inevitable bacterial colonization under the membrane.

Disadvantages of symmetrical hard healing abutments include seating interferences with adjacent hard structures, inadequate direct coverage of adjacent bone grafts, limited membrane location stabilization, lack of a bioseal at the healing abutment-membrane interface, the lack of convenient reconfigurability to closely abut against the gingival perimeter to obtain initial bone graft biosealing or for emergence profile shaping, efficient transmission of oral forces to non-integrated implants, and so on.

Some clinicians utilize a stock hard graft cover that is anchored via an eccentric hole to a hard healing abutment. The hard graft cover may be rotated to a corrected orientation over an otherwise exposed membrane in order to provide a degree of protection for the membrane. The hard graft cover tends to cover over the gingival surface, and over the membrane. The hard graft cover tends to leave substantial portions of the gingival surface and the membrane uncovered, and unsealed. The hard graft cover is not readily reconfigurable toward obtaining a biosealing contact with the gingival perimeter. Further, the eccentric hole is unsealed, and constitutes a bacterial intrusion route to infect the bone graft.

Some clinicians utilize asymmetric hard healing abutments that are custom manufactured to cover an individual bone graft configuration, and to abut against an individual gingival perimeter configuration. The custom manufacture of custom asymmetric hard healing abutments involves careful user planning and designing, complex equipment, and substantially greater time and cost.

Asymmetric hard healing abutments typically cannot be rotationally secured to the implant platform due to potential interferences with adjacent hard structures. As a result, asymmetric hard healing abutments require the use of a titanium-base “ti-base” abutment at the core and separate screw for securing to the implant platform. Ti-base abutments permit vertical, non-rotational, seating onto the platform.

The hard asymmetric healing abutment has a screw access hole for the ti-base screw to pass through. The screw-screw access hole interface is unsealed, and constitutes a bacterial intrusion route to infect the bone graft. The screw access hole in the asymmetric hard healing abutment therefore must be biosealed using an additional and separate procedure, specifically, by placing an occlusal dental filling in the access hole after the screw is secured.

After placement, hard asymmetric healing abutments presents a relatively large surface area for inadvertent oral forces to impinge upon. Such forces are common in the oral environment, and tend to disrupt implant osseointegration.

Further, the perimeter of asymmetric hard healing abutments extend several millimeters to contact the gingival perimeter, which represents a significant lever arm length. The long lever arm amplifies disruptive oral forces transferring to the osseointegrating implant.

Hard asymmetric healing abutments are not readily or conveniently reconfigurable, such as after bone graft stabilization when an ideal emergence profile configuration is needed. To obtain an emergence profile configuration, a custom hard healing abutment typically requires a removal and replacement cycle after reconfiguration or remanufacture. Removal and replacement cycles require additional time, causes site disturbance, risks swallowing or aspirating hard parts, escalates treatment anxiety, permits gingival collapse over the platform, and so on.

In summary, hard healing abutments of the art are inconvenient to reconfigure to fit a gingival perimeter, are not biosealed with added membranes, and transmit traumatic forces to integrating implants.

### SUMMARY

The present invention is directed to placing dental implants, and the placement of bone grafts and healing abutments for the management of gingival and osseous healing.

For the purposes of this discussion, the term “abutment” is used broadly, and includes healing abutments for use during gingival and osseous healing, and also includes abutments that remain secured to an implant after gingival and osseous healing is substantially complete. In some implementations, the term abutment includes an implant temporary prosthesis or final prosthesis.

The terms “facial” and “buccal” are used interchangeably and are not to be construed as limitative. The term “lingual” is intended to include the term “palatal.” The terms “over” and “under” are used as if the dental implant is placed vertically in an osteotomy with the platform facing upward as shown in the drawings, but the terms are not to be construed as limitative. The terms “soft,” “flexible,” and “elastomeric” are used interchangeably. The terms “connected,” “secured,” “contained,” and “biosealed” are used interchangeably.

In the present invention, a healing abutment has a flexible flange extending laterally a distance that is capable of biosealingly containing bone grafts, and forming gingival emergence profiles, and is called a flanged abutment. The flanged abutment at least substantially occupies the space over the implant platform in order to contact the gingiva thereover, and to manage gingival healing after implant placement.

In some implementations, the flanged abutment has a body centered over the implant. The abutment body comprises a volume of material for occupying space over the implant. The shape and volume of the abutment body are provided in various volumes and configurations to enhance fitting the spaces over implants.

In some implementations, the abutment body is comprised of a hard, rigid material. In some implementations, the abutment body is substantially comprised of substantially flexible or elastomeric materials, and is called a soft body. A flanged abutment having a soft body is called a soft flanged abutment. The elastomeric material of the soft body has a sufficiently low durometer such that it readily deforms under a forceful load. When the soft body deforms and flexes under forceful loading, then the ambient forces impinging on the surface of the soft body are substantially absorbed and diffused by the soft body. Such absorption of forces by the soft body reduces forces transferred to the implant that have significant potential to disrupt implant osseointegration. Such forces may be from occlusion of opposing teeth, from debris in the mouth impinging upon the soft body, from extraoral objects impacting the soft body, and so on.

Ambient forces brought to bear against the surface of the soft body are substantially absorbed and diffused. As such, significantly less force is transferred to the implant or to the osteotomy via the soft, flexible, healing abutment.

The soft body is therefore configured to be substantially flexible throughout at least the greater portion of the soft body volume. Body flexibility is greatest for the portion that is distal from the implant platform.

Further, the soft body of the soft flanged abutment lacks the presence of higher durometer, more rigid, materials or structures a small distance under the top surface of the soft body, where the top surface of the soft body is the portion that is distalmost from the implant platform, and generally protrudes above the gingiva. Instead, the flexible material comprising the soft body extends to a substantial depth below the top surface of the soft body.

Soft body healing abutments, or the flanges connected thereto, that have a substantially greater diameter are securable to implant platforms due to a lack of rigid interference with adjacent bony structures or teeth. A rigid interference occurs when a hard abutment forcefully contacts adjacent hard bony structure or adjacent teeth, such as during insertion into an implant platform. Such rigid interferences are known to prevent secure seating of the hard abutment into the implant platform. Soft body abutments and flanges readily deform when forced into contact with an interfering hard structure, called a soft interference. As such, soft body abutments and flexible flanges do not prevent the abutment from securely seating into the implant platform.

As a result of the lack of rigid seating interferences, a soft abutment body permits user to select a larger size soft abutment than they would be able to select for a rigid body abutment. The larger abutments form larger emergence profile spaces, which permits larger diameter prostheses. Larger diameter prostheses are less likely to fracture. A larger diameter soft abutment is also shapable to a more ideal emergence profile shape.

Anatomical, asymmetric, ideal emergence profiles enhance prostheses strength and esthetics. Ideal emergence profiles also improve user access to the implant, and improve patient comfort associated with prosthesis delivery. In some implementations, an anatomical soft abutment and flange is shaped by custom printing. In some implementations, a stock soft abutment and flange is shaped anatomically by manual trimming. In some implementations, the soft abutment and flange is shaped by the addition of other materials to a smaller diameter soft abutment core.

In some implementations, the soft abutment and flange is reshaped by loading a settable addition material into an anatomical form, and holding the form over the soft body and flange until the addition material has set. Such forms may be removable and some remain over the soft abutment until prosthetic delivery.

Advantages of a soft abutment body and flange include reduced hard interferences to abutment seating, reduced likelihood of damaging impact forces transferred to the integrating implant, reduced inertia mass and resulting forces from patient movement, wider healed gingival emergence profiles, wider diameter prosthesis bases for increased fracture resistance, reduced patient pain delivering prostheses, and improved access to implant when delivering prostheses. The term “wider diameter” means that the emergence profile area, or the prosthesis, has a greater cross-sectional area at a given elevation.

In some implementations, flanged abutments are manufactured as stock components. In some implementations, the configuration of flanged abutments are geometrically symmetrical. In some implementations, the configuration of stock components are modifiable by the clinician, such as by manual milling, CNC, material addition, and so on. In some implementations, the configuration of flanged abutments is anatomical and customized to the specific site of an individual patient. In some implementations, flanged abutments of the present invention may be custom manufactured by processes including 3D printing, milling, or custom molding, and such processes may utilize data from 3D scanning, CT imaging, and so on. Suitable flexible materials include silicone, PEEK, rubber, and so on.

A substantially rigid portion of the soft abutment is a connector that connects the soft body portion to the implant platform. In some implementations, the connector is continuous with the soft body, such that the durometer gradients increase in hardness in portions of the soft body that are more proximal to the implant platform. In some implementations, the connector is a separate substantially rigid part that connects to the soft body on the portion of the connector that is located coronally, and connects into the implant platform on the portion of the connector that contacts the platform. Typically, the connector is secured to the platform by engaging and securing to the implant platform threads, such as with a protruding screw, a protruding split peg with flexible halves, a protruding elastomeric peg, and so on.

In some implementations, the connector connects to the soft body by having overhangs and undercuts for the soft body to matingly engage. In some implementations, the connector is adhered to the soft body. In some implementations, the soft body is retained by overmolding processes.

In some implementations, the top surface of the connector has a driver tool internal socket for rotationally engaging a driver tool, such as a hex driver. In some implementations, the top surface of the connector has a driver tool external socket for rotationally engaging a driver tool. In some implementations, other tool engaging means are provided.

In some implementations, at least a portion of the contacting surfaces between the soft body and the connector are biosealed as provided from the manufacturer, such as by overmolding the soft body onto the connector, or with an adhesive layer or other biosealing material layer, or by pressure sandwiching portions of the soft body between portions of the connector, or by welding, and so on. As such, bacteria are substantially blocked from intrusively migrating from the oral environment to the bone graft, and potentially infect the bone graft.

The healing abutment has a flexible flange extending laterally outward from at least a portion of the perimeter of the abutment body. In some implementations, the flange extends radially from the abutment body.

The flange extends a distance laterally from the implant that is sufficient to cover and contain the bone graft. More specifically, the flange extends a distance laterally from the implant that is sufficient to biosealingly contact the gingival perimeter. Where the flange biosealingly contacts the gingival perimeter, then the bone graft is also covered and contained at that location.

Close contact between the flange and the gingival perimeter will form a reliable bioseal that will prevent bacterial intrusion that could infect the bone graft. The biosealing affect between the flange and the gingival perimeter is analogous to the biosealing that occurs when a healing abutment perimeter closely contacts the gingival perimeter. Ambient blood and gingival cellular activity act in concert to achieving a bioseal against the surfaces of the abutment body and against the flange perimeter.

In some implementations, the gingival perimeter will biosealingly contact portions of the abutment body directly that are in close proximity to the gingival perimeter. As such, the flange on that portion is not essential to achieve a bioseal with the gingival perimeter. In some implementations, the healing abutment is preconfigured with the flange absent in that portion of the healing abutment perimeter, or the flange may be trimmed away by the user for that portion. The flexible flange is readily and conveniently trimmable by a user, either while connected to the implant, or extraorally.

In some implementations, the flange extends radially outward from the entire perimeter of the abutment body, such that the flange radius is identical around the entire perimeter of the abutment body. As such, the overall shape of the flanged abutment from a top view is circular with the soft body in the center. In some implementations, the flange extends radially outward from the entire perimeter of the abutment body, such that the flange radius varies over portions of the body perimeter. In some implementations, the overall shape of the flanged abutment from a top view has other geometries, such as ovoid, or rectangular or triangular with rounded angles, irregular, and so on. In some implementations, the abutment body is centrally located within the top view geometric shape. In some implementations, the abutment body is eccentrically located within the top view geometric shape. In some implementations, the healing abutment does not have a body portion that is readily distinguishable from the flange portion.

In some implementations, the flange radius is sufficient to cover and biosealingly contain the entire surface of the bone graft within the bony void over which it is placed. In some implementations, the flange has stock dimensions that can be customized by the user in order to mate portions of the flange radius to the dimensions of the bony void, to the bone graft, and to the gingiva. In some implementations, the flange is custom manufactured for a specific individual bone graft site. In some implementations, the abutment body and the flange are custom manufactured for a specific individual bone graft site.

A flange radius typically would extend laterally 1-15 mm from the body surface, and more specifically 5-10 mm. In some implementations, the flange vertical thickness is from 0.1-2 mm, and more specifically 0.2-0.5 mm. In some implementations, the flange thickness is from 1-7 mm, and more specifically, 3-5 mm.

The flange has a substantial degree of flexibility. The flexibility of the flange facilitates convenient placement adjacent to other structures, such as the adjacent gingiva, teeth, or prostheses. The flexibility also facilitates convenient user customization of the flange radius and shape during placement. However, the flexibility is not excessive such that bone graft particulates are lost beyond an acceptable level, or biosealing was erratic. The flange has sufficient rigidity to maintain a given configuration, such that the flange is able to adequately bioseal the bone graft. In some implementations, portions of the flange have higher rigidity than other portions.

It is an object of the present invention that the flange biosealingly contact the adjacent gingival perimeter. Contact between the gingiva and the flange facilitates biosealing in order to inhibit bacterial intrusions between the gingiva and the flange perimeter, thereby forming a gingival bioseal. Contact between the gingiva and the flange can also control gingival contours, influence the gingival emergence profile, and contain and stabilize bone grafts.

For the purposes of this discussion, bone graft containment, and bone graft biosealing, are closely related, and the terms may be used interchangeably. The relationship between the terms may also be referred to as “biosealingly contained” or “bioseal containment.”

In some implementations, it is an object for the flange to forcefully press against the adjacent gingival borders. Contact between the gingiva and the flange has a similar function as contact between the gingiva and the healing abutment body.

In some implementations, the flange is tucked under the gingiva, and over the outer surface of the alveolar bone immediately surrounding the bony void into which the bone graft is placed, called the perimeter bone. In some implementations, the flange is tucked under the periosteum layer of the gingiva. In some implementations, the flange is tucked into a pouch entirely within the gingiva, such that gingiva surrounds the upper and lower surfaces of the flange perimeter.

In some implementations, it is an object for the flange to substantially remain out of contact with the gingiva, such that a perimeter space is preserved for the introduction of biological materials to enhance bone graft healing, bone graft containment, gingival healing, gingival contouring, and biosealing.

In some implementations, the flange is comprised of the same material as the abutment body to which it is connected. In some implementations, the flange is comprised of a material that is partly or entirely different from the abutment body.

In some implementations, the flange has a similar hardness, or a similar rigidity, with respect to the body of the abutment. In some implementations, the flange has a substantially different hardness or rigidity than the body of the abutment.

In some implementations, the radius of a flange of a single soft abutment is sufficient to cover bone grafts placed around multiple adjacent implants.

In some implementations, the radius of the body of a stock soft abutment extends substantially beyond the margins of at least a portion of the bony void. The user is able to reduce the radius of the soft abutment body, as well as any flange portions, to mate the abutment radius to the configuration of the bony void and the gingival perimeter.

In some implementations, the body of the healing abutment substantially occludes the entire bony void, and has sufficient contact with all surrounding gingiva to contain the bone graft. As such, the body is configured to extend radially a distance away from the implant. In this configuration, the body is in essence functioning as a full-thickness flange. Portions of the body that overextend beyond the bony and gingival margins are conveniently removable by a user.

In some implementations, portions of the flexible flange may be comprised of materials that are different, or heterogenous, from the body, or even than adjacent portions of the flange. Heterogenous flange materials include unexpanded PTFE, poliglecaprone 25, polyglactin 910, polyglycolic acid, silicone, PEEK, rubbers, cellulose, antibacterials, and so on.

In some implementations, portions of the flexible flange, or the body are comprised of various materials, including silicone, silicone blends, PEEK, rubbers, cellulose, antibacterials, absorbables, and so on.

In some implementations, the flange and/or the abutment body are produced by printing, molding, injection molding, overmolding, overmolding onto the base of the healing abutment, and other relevant manufacturing methods.

In some implementations, portions of the flange may have surface textures that are different than adjacent portions of the flange, or portions of the body.

In some implementations, portions of the flange have different structural shapes than adjacent portions of the flange. Examples of flange structural shapes include reinforcing struts, elevated areas, depressed areas, and so on. For example, a locally elevated area may be preferred by a user to create a thickened portion of bone grafting adjacent to the implant.

The flexibility of the flange is such that any interfering contact with hard objects at the perimeter of the bony void during seating of the healing abutment will not prevent full and secure seating of the healing abutment into the implant platform. Hard objects at the perimeter of the bony void include alveolar bone and adjacent teeth. In the event of such an interference with the bony perimeter occurs, the flange is readily deformable by the contact with the hard object, such that further seating of the soft abutment is permitted, and not prevented. The user also has the option to conveniently trim away such interfering portions of the flange at any time before, during, or after seating of the soft abutment into the implant platform. As such, the flange flexibility substantially eliminates seating rigid interferences for a flanged abutment.

The ability to rotate the healing abutment for securing to the implant without encountering hard interferences to rotation or to seating is a significant advantage for flexible flanges, as well as for soft bodies of healing abutments. As such, secure seating can be performed prior to custom fitting the flange to the site.

In some implementations, the flange is connected homogenously with the body, so that the flange is integral with the body. In some implementations, a flange material is printed continuously with a heterogenous body material. In some implementations, a separate flange is connected to the body, such as by engagement into an undercut or groove in the body, by anchoring means, by sandwiching between the body and the abutment connector, by engaging into the abutment connector, by engaging a separate screw, and so on. In some implementations, the flange is attachably detachable from the body, or from the abutment connector. In some implementations, the connection of the flange to the healing abutment body is provided biosealed, such as with adhesive, caulking, sandwiching, and so on.

In some implementations, a detachable connectable flexible flange, called a detachable flange, is connected to a healing abutment body when the healing abutment is secured to an implant platform. In some implementations, the detachable flange is symmetrical. In some implementations, the detachable flange is asymmetrical. In some implementations, the detachable flange is fabricated in stock root-form shapes. In some implementations, the detachable flange is readily reconfigurable by a user, such as for fitting over a bony void.

In some implementations, a detachable flange may be connectable to a healing abutment prior to securing the healing abutment to an implant platform. In some implementations, a detachable flange is removable from connection to a healing abutment without requiring removal of the healing abutment from the implant platform.

In some implementations, the flange has surface features for enhancing connectivity to healing gingiva or to added bone graft covers. Surface features include small dimples, slots, recesses, undercuts, spikes, hooks, papules, pedunculated papules, pores, roughness, and so on. In further descriptions, all such surface features are called “slots.”

Slots may cover the entire upper or lower surfaces of the flange, or be restricted to specific surface areas. Sizes and arrangements of slots may be uniform or non-uniform.

Slots are primarily for the engagement, retention, or adhesion to the gingival perimeter, or to biomaterials introduced to enhance bio-sealing the perimeter against the intrusion of bacteria, and for managing gingival healing.

It is an object of the present invention that flange durometer, configuration, and slots enhance formation of a bacterial bioseal around the perimeter of the abutment body at the body-flange interface, and at the flange-gingival perimeter interface.

Other biomaterials may be added to contact the flange or abutment to form or enhance the bioseal, such as bone graft cover materials, called a graft cover. However, the flanged abutment is capable of forming a bioseal with the gingival perimeter due to ambient blood and gingival cellular connectivity, and without any added graft cover.

In some implementations, a graft cover is able to enhance the reliability or stability of flanged abutment biosealing or gingival shaping. The flange securely engages a graft cover, and thereby stabilizes the location and configuration of the graft cover. Flange stabilization of a graft cover facilitates bone graft containment, biosealing of the bone graft, biosealing the gingival perimeter, and shaping the gingival perimeter.

In some implementations, the addition of a graft cover to a flange is considered to be a means of shaping the flange, or a means of shaping the flanged abutment body, in order to enhance a bioseal, or form a preferred gingival emergence profile. In some implementations, a graft cover is added to a flange in order to refine the configuration and fit of the flange with the gingival perimeter.

Graft cover materials include materials known in the art to contain bone grafts, to enhance bone graft healing, or that enhance a bioseal to prevent bacteria from infecting a bone graft site. Graft cover materials include PTFE, d-PTFE, hydroxyapatite, PRF, exosomes, ambient blood, growth factors, periodontal dressings, periodontal packs, free gingival grafts, collagen particles, collagen, collagen membrane, PeriAcryl, poliglecaprone 25, polyglactin 910, polyglycolic acid, temporary cements, impression materials, and so on, used alone or in combination. Ambient blood typically coats the interfaces of the flange, graft cover, membrane, and gingiva, and is the most important graft cover for biosealing.

In some implementations, graft covers are pliable or flowable prior to placement, and remain at least pliable after placement. In some implementations, graft covers are pliable or flowable prior to placement, and are substantially hardened after placement, such as by reaction of parts A/B, or by exposure to saliva, or by light curing, and so on.

In some implementations, a flange extends radially from the abutment body at a vertical elevation that is approximately level with the surface of a typical bone graft placement, called a bone-level flange. As such, the flange undersurface would cover the bone graft, and substantially contact the bone graft. The flange elevation typically would be 0-3 mm above the implant platform, and more specifically, 1-2 mm above the platform. The user would typically trim away overextended portions of the midlevel flange that would substantially impinge on adjacent teeth or gingiva.

In some implementations, a membrane type of graft cover is biosealingly pre-connected to the lower surface of the bone-level flange, and to the slots thereon. In some implementations, a graft cover is placed and biosealingly retained over the upper surface of the bone-level flange having slots.

In some implementations, a flange that extends radially from the abutment body at a vertical elevation that would be substantially above the surface of a typical bone graft placement, but still below the level of the gingival surface, is called a midlevel flange. As such, the midlevel flange would be out of direct contact with much of the bone graft. The midlevel flange would be located sufficiently off the bone graft such that a graft cover could be placed and biosealingly retained in the space between the bone graft surface and the under-surface of the midlevel flange.

The elevation of a midlevel flange typically would be 1-6 mm from the implant platform, and more specifically, 2-4 mm above the platform. The user would typically trim away overextended portions of the midlevel flange that would substantially impinge on adjacent teeth or gingiva.

In some implementations, the midlevel flange has slots configured to retain bone graft retention materials. In some implementations, graft cover materials would be placed both under and over portions of the midlevel flange surfaces. As such, bone grafts could flow into midlevel flange retention slots from above and below the midlevel flange, for enhanced retention to the midlevel flange, and for enhanced biosealing to restrict bacterial entrance from around the flange perimeter.

In some implementations, a graft cover material is pre-adhered to the midlevel flange. In some implementations, a membrane is pre-connected to the midlevel flange, and to the slots thereon.

In some implementations, a flange is located at an elevation near the top of the body, and level with the perimeter gingiva surface, and is called a tissue-level flange. A tissue-level flange is able to cover over the surface of the perimeter gingiva. Graft cover materials added under the tissue-level flange would be substantially contained thereby. The user would typically trim away tissue-level overextended flange portions, such as portions that interfered with adjacent teeth after insertion over the site. A tissue-level flange elevation would be from 2-10 mm above the implant platform, and more specifically, 4-7 mm above the platform.

In some implementations, a flange thickness approximates the thickness of the surrounding gingiva, and is called a full-thickness flange. A full-thickness flange is readily user-trimmable so as to be abuttable against the surrounding gingiva, and to adequately cover bone graft. The full-thickness flange inhibits the gingiva from healing over the flange. As such, the shape of the full-thickness graft can effectively define the gingival emergence profile.

When the gingiva is substantially in contact with the full-thickness flange, it is anticipated that ambient blood will substantially form a bioseal in the small contact space between. This is analogous to the way that the gingiva bioseals against the surfaces of a healing abutment body. The presence of open slots along the vertical surface of the full-thickness flange can facilitate biosealing with ambient blood, wherein the blood can flow into, and be retained by the slots.

In some implementations, a full-thickness flange has slots for retention of graft cover materials and perimeter biosealing materials. In some implementations, slots are manufactured so as to be located throughout the thickness of the full-thickness flange, so that initially unexposed internal area of the flange contains a multiplicity of slots.

In some implementations, a full-thickness flange is manufactured with a graft cover connected to the lower surface where it would be in contact with the bone graft. In some implementations, a full-thickness flange is manufactured with a collagen membrane-type graft cover connected to the lower surface where it would be in contact with the bone graft. In some implementations, a full-thickness flange is manufactured with an unexpanded d-PTFE membrane-type graft cover connected to the lower surface where it would be in contact with the bone graft.

Users are able to trim the flange and the attached membrane to a first dimension. As such, the membrane and the flange both cover all the bone graft surface at the initial implant and bone graft placement surgery. The membrane further facilitates flange biosealing against the gingiva.

After bone graft stabilization, the user may further trim the membrane graft cover and flange to a second dimension to facilitate formation of an ideal gingival emergence profile, thereby leaving a portion of the stabilized bone graft uncovered. The gingiva would be expected to grow over the exposed bone graft until it comes into contact with the flange and graft cover.

In some implementations, a biosealingly pre-attached membrane-type graft cover may be partly pulled away from the lower surface of the full-thickness graft to facilitate trimming the membrane to a first dimension and trimming the flange to a second dimension. As such, the remainder of the membrane remains biosealingly attached to the flange. Users are thereby able to trim the membrane to a dimension that covers all the exposed bone graft, while trimming the flange to a dimension that defines the gingival emergence profile during the initial implant and bone graft placement surgery.

At initial bone graft placement, in some implementations, a full-thickness flange is user-trimmed to cover the entire bone graft, and securely abut against all surrounding gingiva, or at least the gingiva that is not abutted by the body. In some implementations, after a time period when the bone graft has had sufficient time to stabilize, a full-thickness flange is readily user-trimmed further, so as to expose the stabilized bone graft. As such, the full-thickness flange is trimmed to form a gingival emergence profile desired to facilitate prosthetic design.

In some implementations, a first full-thickness flange and abutment body are removed from the connector after bone graft stabilization, and replaced by a second full-thickness flange and body that are configured to form an ideal gingival emergence profile. In some implementations, the second full-thickness flange with body is a stock item, and in some implementations, second full-thickness flange with body is custom manufactured.

In some implementations, a first flange is detachably connected to an abutment. After bone graft stabilization, a second flange is then detachably connected to the abutment, such as to facilitate formation of an ideal gingival emergence profile.

In some implementations, a flange is biosealingly connected to an abutment by compression between the abutment and the implant platform walls. In some implementations, the platform and the flange have mating Morse taper configurations to further facilitate a biosealing connection.

In some implementations, a soft abutment body contains a generally toroidal space around the driver access, called the toroid. The toroid is expandable, such as by inflation, such that a flange is formed that protrudes laterally from the body over the bone graft. The flange is generally expandable laterally until biosealing contact is made with the gingival perimeter. Toroidal expansion tends to be substantially symmetrical.

Toroidal inflation is typically accomplished by needle penetration through the body top surface and into the toroid to inject pressurized fluids or gas. At least portions of the toroid has a thickened upper surface that functions as a rubber stopper to prevent the leakage of injected fluids or gas. The outer perimeter wall of the toroid is thinner relative to the inner wall adjacent to the driver access. As such, the diameter of the driver access is substantially maintained during toroidal inflation.

In some implementations, the toroid contains a multiplicity of inflatable bladders. Inflating a single bladder cause a limited section of the toroidal perimeter to expand. The sections of the toroidal perimeter that are not containing an inflated bladder substantially remain unexpanded. The toroidal perimeter may be controllably expanded in limited sections so that the flange formed can sealingly contact an entire gingival perimeter that varies in distance from the body. Such sectional control of flange expansion thereby avoids excessive expansive pressures against portions of the gingival perimeter that are closer to the body. Controlled expansion of limited sections of the flange facilitates the formation of appropriate biosealing contact pressures along the entire gingival perimeter.

In some implementations, the wall of a first bladder is not connected to the wall of a second adjacent bladder, so that the walls readily slidingly move relative to one another. As such, the expansion of the individual bladders is substantially unimpeded by contact friction.

In some implementations, the bladders have a thickened upper surface that functions as a rubber stopper to prevent the leakage of injected fluids or gas. In some implementations, a gap is preserved between adjacent bladders, such that a user can generally inflate the toroid to a given diameter, while having the option to independently inflate individual bladders to further increase the body radius in limited sections.

In some implementations, the toroid, the bladders, or both, have expandable bellows that facilitate expansion during pressurization. In some implementations, the lateral exterior wall of the toroid has a degree of rigidity that facilitates forming an ideal gingival emergence profile, such that the rigidity limits stretching and expansion, but provides configuration control and predictability.

In some implementations, the body top surface has visible marks to identify the locations of individual bladders contained within. The marks assist users to locate accurate needle placement during injection of fluids or gasses through the body top surface and into the toroid, or into specific bladders. The body top surface can similarly be marked to identify the location of the gap.

In some implementations, the bladders contain a marker substance to indicate to the user that a needle is correctly positioned within the lumen of a bladder, and ready for subsequent inflating injection. An example of such a marker substance is a colored liquid that may be aspirated from the bladder into a syringe, and thereby indicate correct needle placement for injection.

It is an object of the present invention that the flexible flange is readily alterable, or reconfigurable, while remaining connected to the implant platform. As such, a readily reconfigurable flange facilitates convenient and efficient management of interferences with adjacent structures, gingival healing, gingival emergence profiles, biosealing, and the ability to rapidly respond to ongoing dynamic changes during the healing period.

### BRIEF DESCRIPTION OF DRAWINGS

FIG. 1 is a cross-sectional view of a flanged abutment having a body, a bone-level flange, a connector, and an implanted implant.

FIG. 2 is a perspective view of a flanged abutment having a body, a trimmed bone-level flange, and a connector.

FIG. 3 is a cross-sectional view of a flanged abutment having a body, a trimmed midlevel flange, a connector, and an implanted implant.

FIG. 4 is a cross-sectional view of a flanged abutment having a body, a trimmed tissue-level flange, a connector, and an implanted implant.

FIG. 5A is a cross-sectional view of a flanged abutment having a body, a full-thickness flange configured for containing an unstable bone graft, a connector, and an implanted implant.

FIG. 5B is a cross-sectional view of a flanged abutment having a body, a full-thickness flange configured for forming an ideal gingival emergency profile, a connector, and an implanted implant.

FIG. 6 is a cross-sectional view of a flanged abutment having a body, a detachable flange, a connector, and an implanted implant.

FIG. 7 is a cross-sectional view of a flanged abutment having a body, a compression flange, a connector, and an implanted implant.

FIG. 8A is a cross-sectional view of a flanged abutment having a body, a toroid, uninflated bladders, a connector, and an implanted implant.

FIG. 8B is a cross-sectional view of a flanged abutment having a body, a toroid, inflated bladders, a connector, and an implanted implant.

FIG. 8C is a top view of a flanged abutment having a body, a toroid, uninflated bladders, a connector, and an implanted implant.

FIG. 8D is a top view of a flanged abutment having a body, a toroid, inflated bladders, a connector, and an implanted implant.

### REFERENCE NUMERALS IN DRAWINGS

20A-H flanged abutment
22 implant
24A-H body
26A bone-level flange
26B midlevel flange
26C tissue-level flange
26D full-thickness flange
26E profile flange
26F detachable flange
26G compression flange
26H expandable flange
28 bone graft
30 osseous void
32A-H slots
34 gingiva
36A-D connector
38 platform
40A-D socket
42A-F driver access
44 membrane
46 graft cover
48A-B snap
50 bone
52 groove
54 pellicle
56 toroid
58A-B bladder
60 gap

### DETAILED DESCRIPTION

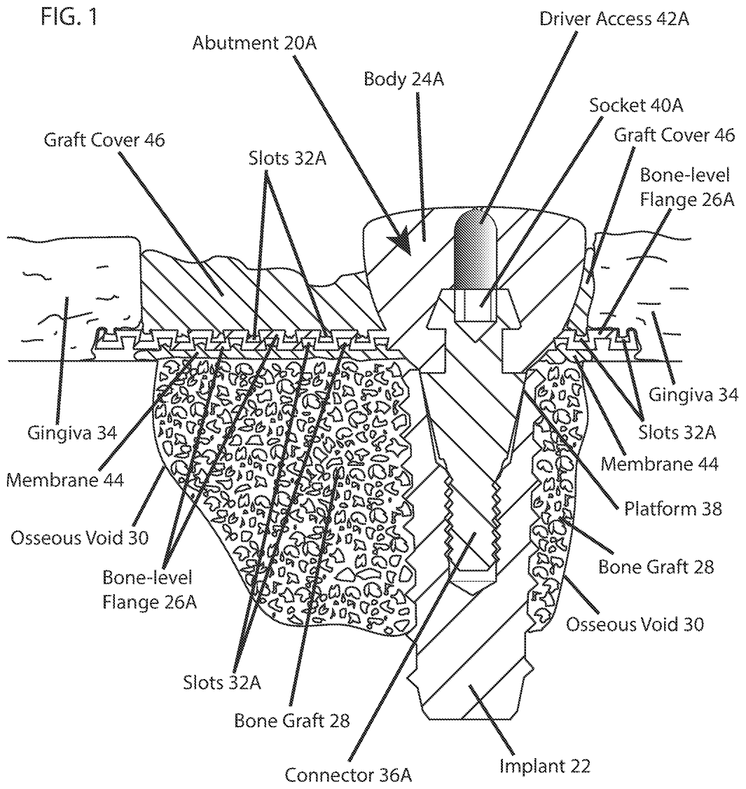

In accordance with an aspect and referring to FIG. 1, there is provided an abutment 20A for securing to an implant 22. Abutment 20A has a body 24A. In some implementations, body 24A is substantially flexible. The elastomeric material of a flexible, soft body 24A has a sufficiently low durometer such that it readily deforms under a forceful load. When a soft body 24A deforms and flexes under load, ambient forces impinging on the surfaces thereof are substantially absorbed and diffused by a soft body 24A.

A bone-level flange 26A, extends radially from body 24A. Bone-level flange 26A is selected to have a sufficiently large radius so as to be readily configurable to substantially biosealingly contain bone graft 28 placed into osseous void 30. Slots 32A are present on bone-level flange 26A for enhancing a bioseal with perimeter gingiva 34. Bone-level flange 26A is shown after user trimming to an individual asymmetric geometry to precisely fit over bone graft 28, and to abut against gingiva 34. In some implementations, bone-level flange 26 is provided with an asymmetric geometry.

A connector 36A retains body 24A with overhangs and undercuts. Connector 34A has a unitary screw portion for securing into platform 38 of implant 22, and a socket 40A for engaging a driver tool. Body 24A has a driver access 42A, which comprises a through-hole for providing access of a driver tool to socket 40A.

A membrane 44 is between bone-level flange 26A and bone graft 28. At least a portion of membrane 44, or an associated adhesive, is shown retentively engaging slots 32A. Membrane 44 facilitates location stabilization of bone graft 28, and facilitates biosealing of bone graft 28.

In some implementations, membrane 44 is trimmed so as to extend to a greater radial diameter than bone-level flange 26A. As such, membrane 44 may be tucked under a small pouch formed under gingiva 34 to further enhance biosealing of bone graft 28.

Graft cover 46 material substantially covers bone-level flange 26A. Portions of graft cover 46 is shown having flowed into, and being retentively engaged by, slots 32A of bone-level flange 26A. The perimeter edge of bone-level flange 26A is user-trimmed to contact gingiva 34, thereby forming a secondary perimeter of bone-level flange 26A. During trimming, a multiplicity of slots 32A are exposed. In some implementations, graft cover 46 retentively engages exposed perimeter slots 32A to enhance biosealing of bone graft 28. In some implementations, graft cover 46 and membrane 44 are comprised of identical materials. After a short time, gingiva 34 is able to directly biosealingly engage bone-level flange 26A and slots 32A, whether or not graft cover 46 is present.

After a sufficient healing period, graft cover 46 and bone-level flange 26A are trimmable by a user, such as to reshape to an ideal gingival emergence profile configuration. Bone-level flange 26A is trimmable without removing abutment 20A from platform 38.

In some implementations, flexible flange 26A is comprised of a very thin, flexible, layer of a high-durometer material, such as a plastic or metal that would otherwise be rigid when provided having a greater thickness. In some implementations, flexible flange 26A is comprised of a thin layer of titanium that is continuous with a titanium body 24A.

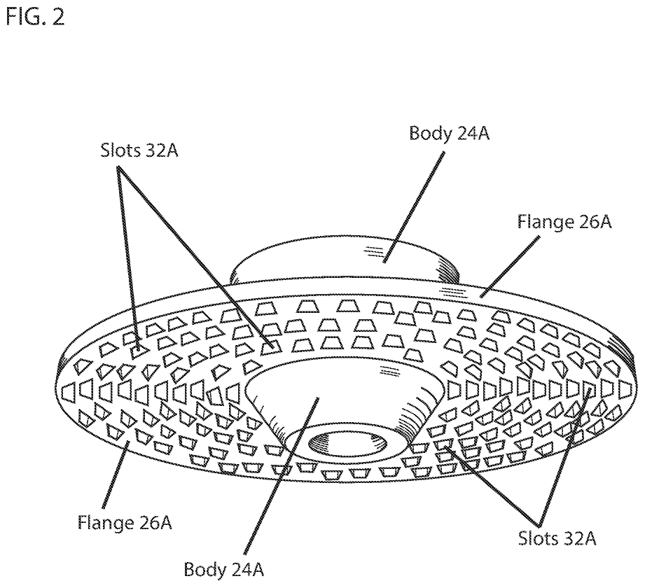

In accordance with an aspect and referring to FIG. 2, there is provided a body 24A with a bone-level flange 26A extending radially from the perimeter of body 24A. The unidentified hole in the bottom of body 24A is for receiving a connector 36A, as shown in FIG. 1. The overall geometry of the body 24 located centrally within bone-level flange 26A would be symmetrical and circular if shown in a top view.

A multiplicity of slots 32A are shown spaced apart at intervals over the lower surface of flange 26A. Slots 32A are also spaced apart along the upper surface of flange 26A, but not shown.

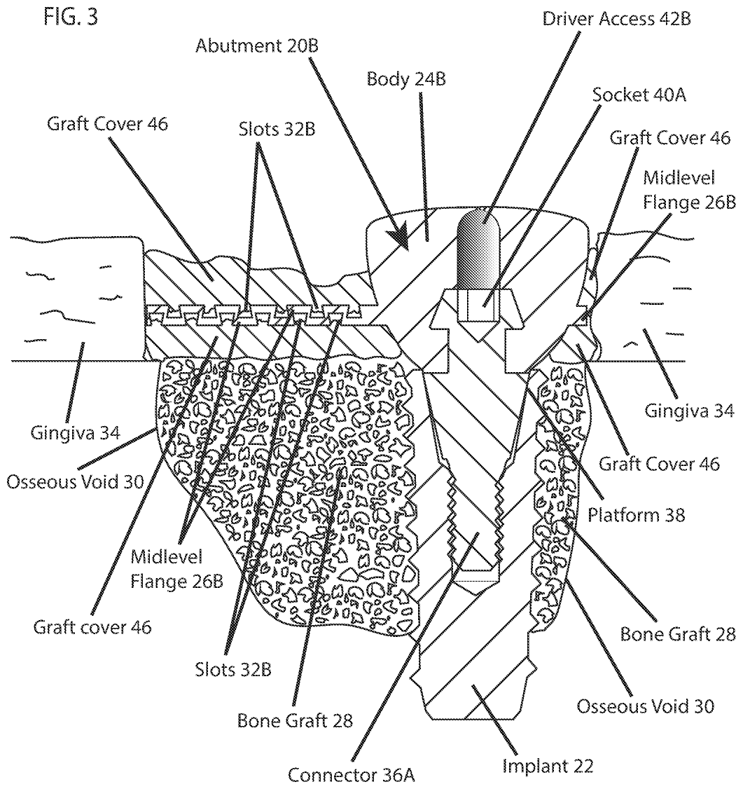

In accordance with an aspect and referring to FIG. 3, there is provided an abutment 20B for securing to an implant 22. A connector 36A connects body 24B to platform 38 of implant 22. A driver access 42B provides access for a driver tool into socket 40A.

A midlevel flange 26B extends radially from body 24B. Midlevel flange 26B is selected to have a sufficiently large radius so as to be readily trimmable to biosealingly abut against gingiva 34. Slots 32B are interspersed over the upper and lower surfaces of midlevel flange 26B, and are retentive of graft cover 46 shown having flowed partly into slots 32B, and biosealingly retained thereby.

Graft cover 46 is shown under midlevel flange 26B such that bone graft 28 in osseous void 30 is substantially covered and biosealingly contained by graft cover 46. Graft cover 46 is also shown on the upper surface of midlevel flange 26B. Graft cover 46 that is both over and under midlevel flange 26B biosealingly contacts gingiva 34, further enhancing the biosealing of bone graft 28.

The perimeter edge of midlevel flange 26B is user-trimmed to contact gingiva 34, thereby forming an altered perimeter of midlevel flange 26B. During trimming, a multiplicity of slots 32B are exposed. Gingiva 34 and graft cover 46 are able to retainingly engage exposed perimeter slots 32B, and thereby further enhance biosealing of bone graft 28. After a short time, gingiva 34 is able to directly biosealingly engage midlevel flange 26B and slots 32B, whether or not graft cover 46 is present.

After a sufficient healing period, graft cover 46 and midlevel flange 26B are user trimmable to form an ideal gingival emergence profile configuration. Midlevel flange 26B is readily trimmable without removing abutment 20B from platform 38.

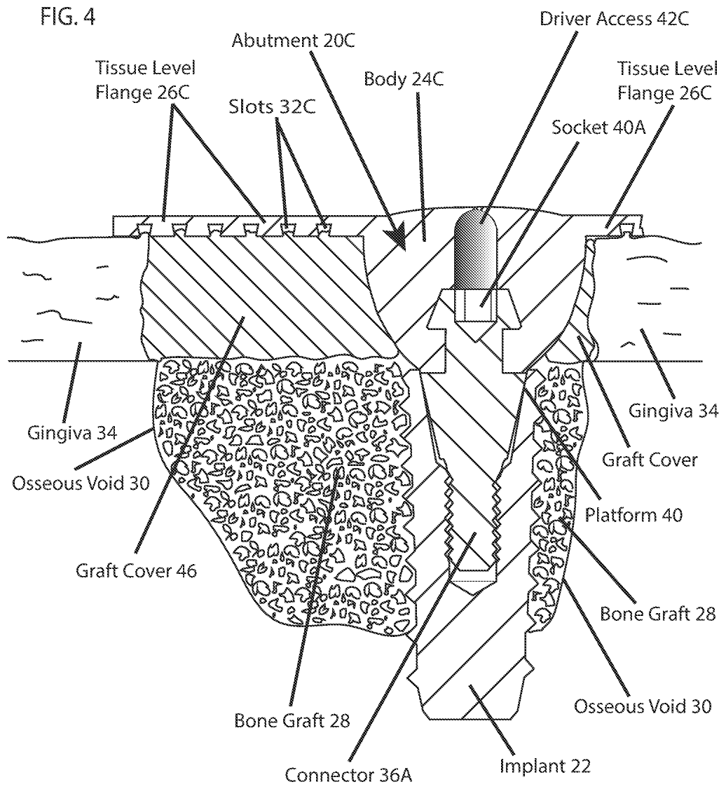

In accordance with an aspect and referring to FIG. 4, there is provided an abutment 20C for securing to an implant 22. A connector 36A connects body 24C to platform 38 of implant 22. A driver access 42C provides access for a driver tool into socket 40A.

A tissue-level flange 26C extends radially from body 24C. Tissue-level flange 26C is selected to have a sufficiently large radius so as to be readily trimmable to overlap the gingiva 34 perimeter. Slots 32C that are interspersed over the lower surface of tissue-level flange 26C are retentive of graft cover 46 shown having flowed partly into slots 32C. Gingiva 34 engages into slots 32C to biosealingly contain bone graft 28.

Graft cover 46 is shown under tissue-level flange 26C such that bone graft 28 in osseous void 30 is substantially biosealingly contained by graft cover 46. Graft cover 46 is substantially retained by tissue-level flange 26C over bone graft 28 and against gingiva 34. After a short time, gingiva 34 biosealingly engages tissue-level flange 26C and slots 32C, whether or not graft cover 46 is present.

After a sufficient healing period, graft cover 46 and tissue-level flange 26C are trimmable by a user, such as to reshape to an ideal gingival emergence profile shape. Tissue-level flange 26C is readily trimmable without removing abutment 20C from platform 38.

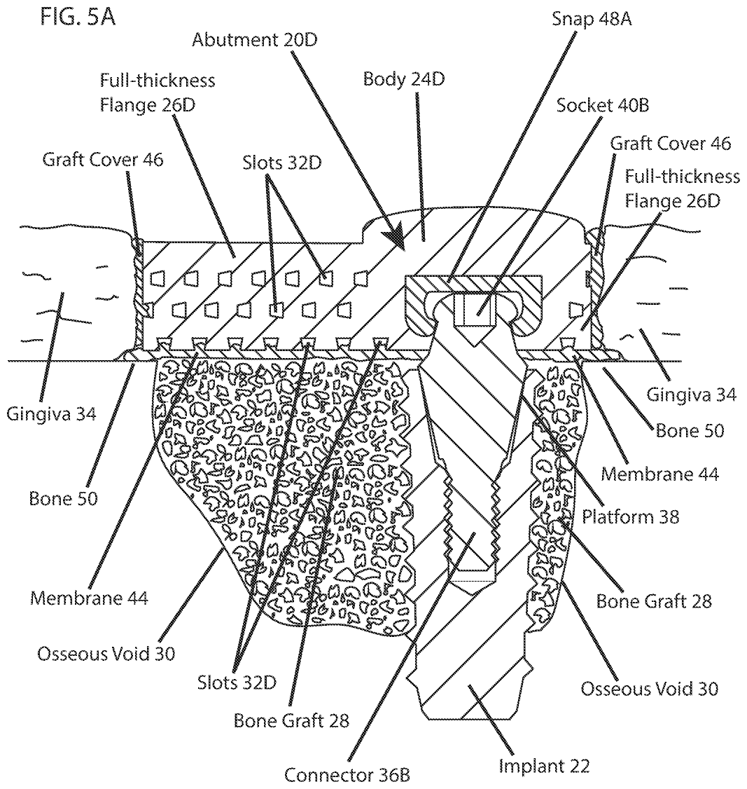

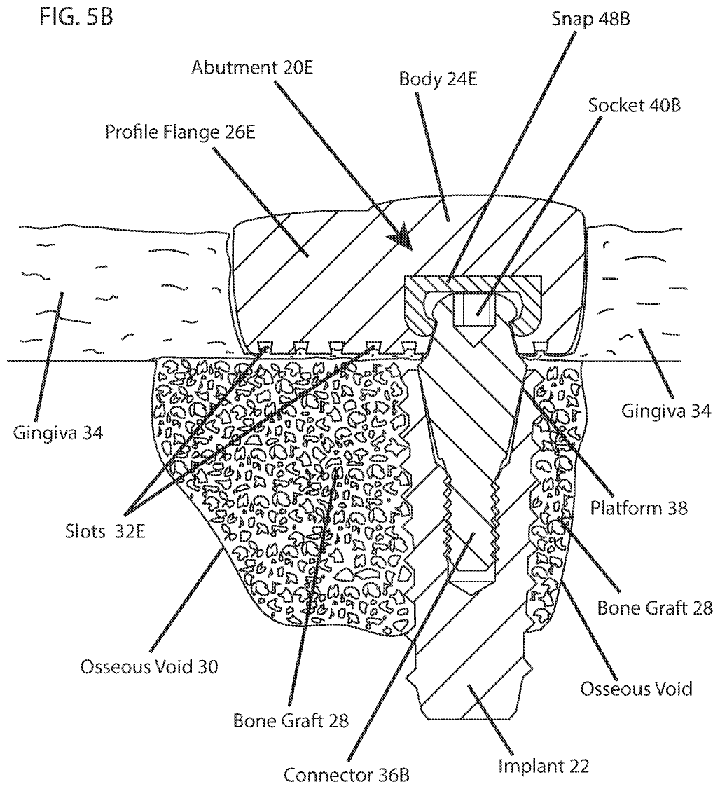

In accordance with an aspect and referring to FIG. 5A, there is provided an abutment 20D for securing to an implant 22. A connector 36B connects body 24D to platform 38 of implant 22. Connector 36B has a threaded screw-portion for securing to platform 38. Connector 36B has a socket 40B for the engagement of a driver tool used to rotationally secure connector 36B to platform 38.

Connector 36B has an undercut for retentively engaging a snap 48A. Snap 48A engages the undercuts of connector 36B when downwardly extending arms of snap 48A flex outwardly away from connector 36B in response to pressure applied to the upper surface of snap 48A. Once the arms of snap 48A move downwardly beyond the height of contour of the connector 36B undercut, then the arms flex inwardly toward the undercuts of connector 36B, thereby permitting snap 48A to retentively fully seat down onto connector 36B. Snap 48A is thereby retained by connector 36B when seated thereon. With snap 48A and body 24D, bacteria are biosealed from intruding down from the oral cavity and passing through the snap 48A connection with connector 36B.

The upper and outward surfaces of snap 48A are retentively connected to body 24D, such as by adhesion, surface roughness, undercuts, overmolding, or other retention means. Sufficient pressure applied to the upper surface of body 24D in turn applies sufficient pressure to the upper surface of snap 48A, such that snap 48A flexes over the undercut height of contour of connector 36B to retentively engage connector 36B.

A full-thickness flange 26D extends laterally from body 24D. Full-thickness flange 26D is selected to have a sufficient lateral extension so as to be readily user trimmable to biosealingly abut against the gingiva 34 perimeter.

Slots 32D are interspersed throughout the entire thickness and width of full-thickness flange 26D. A multiplicity of interspersed slots 32D are exposed along the lateral perimeter of full-thickness flange 26D as provided, prior to trimming or reconfiguration for biosealing engagement with gingiva 34.

Slots 32D interspersed over the lower surface of full-thickness flange 26D are retentive of membrane 44, shown partly engaging into slots 32D. Membrane 44 is shown under full-thickness flange 26D such that bone graft 28 in osseous void 30 is biosealingly contained by membrane 44.

The trimmed perimeter of membrane 44 is shown extending outwardly laterally further than the trimmed perimeter of full-thickness flange 26D. The perimeter edge of membrane 44 is shown tucked into a small pouch created by the user under gingiva 34, and over bone 50, thereby enhancing biosealing of bone graft 28.

The perimeter edge of full-thickness flange 26D is reconfigured by user-trimming to biosealingly contact the gingiva 34 perimeter. During trimming, a multiplicity of slots 32D are exposed. Graft cover 46 placed between gingiva 34 and the perimeter of full-thickness flange 26D can retentively engage exposed perimeter slots 32D, further enhancing biosealing of bone graft 28. After a short time, gingiva 34 can directly biosealingly engage full-thickness flange 26D and slots 32D, whether or not graft cover 46 is present.

In some implementations, after a sufficient healing period, graft cover 46 and full-thickness flange 26D are trimmable by a user to form an ideal gingival emergence profile configuration. Full-thickness flange 26D is readily trimmable without removing abutment 20D from platform 38.

In accordance with an aspect and referring to FIG. 5B, there is provided an abutment 20E for securing to an implant 22. A connector 36B connects body 24E to platform 38 of implant 22. Connector 36B has a threaded screw-portion for securing to platform 38. Connector 36B has a socket 40B for the engagement of a driver tool used to secure connector 36B to platform 38.

Connector 36B has an undercut for retentively engaging a snap 48B. Snap 48B engages the undercuts of connector 36B when downwardly extending arms of snap 48B flex outwardly away from connector 36B in response to pressure applied to the upper surface of snap 48B. Once the arms of snap 48B move downwardly beyond the height of contour of the connector 36B undercut, then the arms flex inwardly toward the undercuts of connector 36B, thereby permitting snap 48B to retentively fully seat down onto connector 36B. Snap 48B is thereby retentive to connector 36B when seated thereon. With snap 48A and body 24E, bacteria are biosealingly prevented from intruding down from the oral cavity and passing through the snap 48A connection with connector 36B.

The upper and outward surfaces of snap 48B are retentively connected to body 24E, such as by adhesion, surface roughness, undercuts, overmolding, or other retention means. Sufficient pressure applied to the upper surface of body 24E in turn applies sufficient pressure to the upper surface of snap 48B, such that snap 48B flexes over the undercut height of contour of connector 36B to retentively engage connector 36B.

A profile flange 26E extends a distance laterally from body 24E and connector 36B. In some implementations, the top view of profile flange 26E is non-radial, and non-symmetrical. Profile flange 26E is selectable to have, or can be reconfigured to, a perimeter configuration that facilitates the formation of an ideal gingiva 34 emergence profile. Further, abutment 20E is selectable to have, or can be reconfigured to, a perimeter configuration that facilitates the formation of an ideal gingiva 34 emergence profile.

Gingiva 34 is shown after a degree of healing and growth since implant 22 placement. Gingiva 34 has substantially covered stabilized bone graft 28. Gingiva 34 is biosealingly engaging slots 32E to substantially prevent bacterial infection along the interface between profile flange 26E and gingiva 34. Gingiva 34 is contacting the lateral surfaces of profile flange 26E, so as to form an ideal emergence profile configuration of gingiva 34.

In some implementations, profile flange 26E comprised of a low durometer material that is identical to body 24E. In some implementations, profile flange 26E has durometer and material composition that differs from body 24E. A low durometer profile flange 26E is substantially able to absorb oral forces that could interfere with osseointegration of implant 22.

In some implementations, body 24E with profile flange 26E are readily rotatable before or after snap 48B is secured to connector 36B, such that the ideal rotation is readily obtainable. In some implementations, snap 48B resists inadvertent rotation of body 24E and profile flange 26E with respect to connector 36B.

Snap48B is concentric with connector 36B and implant 22. However, in most cases, the overall lateral configuration of abutment 20E will be asymmetric in order to fit asymmetric placements of implants 22 relative to the ideal gingiva 34 emergence profile.

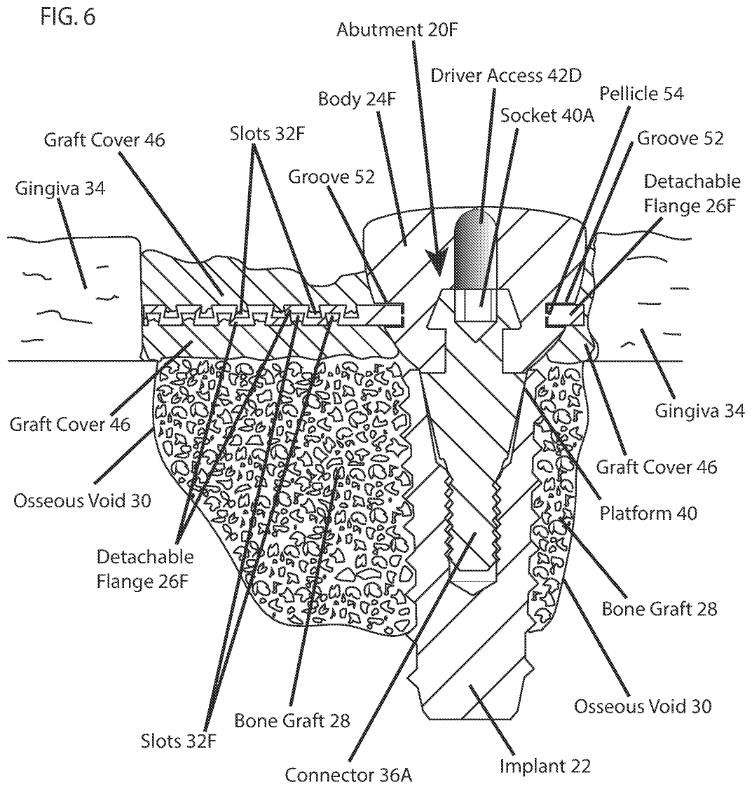

In accordance with an aspect and referring to FIG. 6, there is provided an abutment 20F for securing to an implant 22. A connector 36A connects body 24F to platform 38 of implant 22. A driver access 42D provides access for a driver tool into socket 40A.

A detachable flange 26F extends radially from body 24F. Detachable flange 26F has an inner perimeter that abuts against body 24F. Detachable flange 26F is detachably and securely retained to body 24F by a retentive feature on the surface of body 24F, such as a lessor diameter groove 52 in the surface thereof. As such, the inner perimeter of detachable flange 26F is in mating contact with the surfaces of groove 52.

In some implementations, detachable flange 26F is comprised of elastomeric material, and undergoes elastic deformation while engaging groove 52. As such, detachable flange 26F forms an elastomeric pressure fit into groove 52, which eliminates small and micro-gaps between interfacing surfaces. Detachable flange 26F biosealingly contacts groove 52, which is facilitated by ambient blood.

A pellicle 54 biosealingly detachably retains detachable flange 26F in groove 52. Pellicle 54 is continuous between body 24F and detachable flange 26F. Pellicle 54 is a thin connecting membrane or flange that tears readily to facilitate intentional removal of detachable flange 26F.

In some implementations, other biosealing retentive means for detachably retaining detachable flange 26F may include ambient blood, adhesive, caulking, pins, pinching detachable flange 26F between divided separate portions of body 24F, and so on.

Detachable flange 26F is selected to have a sufficiently large radius so as to be readily configurable to biosealingly abut against gingiva 34. Slots 32F are interspersed over the upper and lower surfaces of detachable flange 26F, and are retentive of graft cover 46 shown having flowed partly into slots 32F. A variety of detachable flange 26F configurations may be provided to enhance ease of use, retention of biosealing material, gingival emergence profile, and so on. After a short time, gingiva 34 is able to directly biosealingly engage profile flange 26F and slots 32F, whether or not graft cover 46 is present.

In some implementations, a first detachable flange 26F is provided preconnected to abutment 20F, which may later be detached, such as after stabilization of bone graft 28. After a first detachable flange 26F is detached from abutment 20F, then a second detachable flange 26F may be connected via a portion that engages groove 52 for forming an ideal gingiva 34 emergence profile. The configuration of a second detachable flange 26F is selectable or configurable to facilitate the formation of an ideal gingiva 34 emergence profile.

Graft cover 46 is shown under detachable flange 26F engaging slots 32F, such that bone graft 28 in osseous void 30 is biosealingly contained. Graft cover 46 is also shown on the upper surface of detachable flange 26F engaging slots 32F. Graft cover 46 that is both over and under detachable flange 26F biosealingly contacts gingiva 34, and bioseals bone graft 28. After a short time, gingiva 34 is able to directly biosealingly engage detachable flange 26F and slots 32F, whether or not graft cover 46 is present.

In some implementations, detachable flange 26F is located at various elevations with respect to body 24F, including at bone-level, at tissue-level, or other preferred levels. In some implementations, various vertical thickness of detachable flange 26F are provided to facilitate convenient bone graft 28 biosealing or gingival 34 emergence profile formation.

In some implementations, a hard, high-durometer body 24F may be utilized. In some implementations, an elastomeric detachable flange 26F may similarly be detachably connected to a hard body 24F for a given time period during the healing of bone graft 28.

In some implementations, a moldable settable detachable flange 26F material is placed to biosealingly contain bone graft 28 or to form a gingiva 34 emergence profile, wherein the moldable settable detachable flange 26F material is at least partly retained by retentive features on body 24F. The moldable settable detachable flange 26F is user-attachable to body 24F, and is user-configurable for custom fitting to an individual site. Suitable materials for moldable settable detachable flange 26F include graft cover 46, 3D printed materials, and so on.

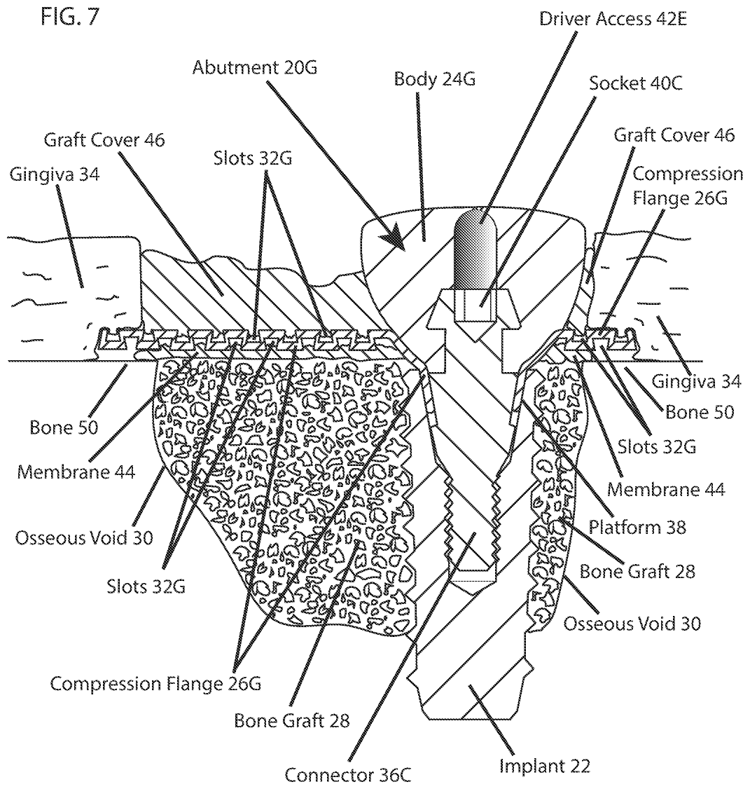

In accordance with an aspect and referring to FIG. 7, there is provided an abutment 20G for securing to an implant 22. A connector 36C connects body 24G to platform 38 of implant 22. A driver access 42E provides access for a driver tool into socket 40C. A compression flange 26G connects directly to connector 36C, and extends radially from connector 36C. Compression flange 26G has a central through-hole for receiving connector 36C.

In some implementations, compression flange 26G is a manufactured as a continuous extension of connector 36C. In some implementations, compression flange 26G is comprised of a material that is identical to the material of connector 36C. In some implementations, compression flange 26G is manufactured separately from connector 36C, and is preconnected to connector 36C prior to seating into platform 38. In some implementations, connection between compression flange 26G and connector 36C is formed by compressive friction created when compression flange 26G is pressed onto connector 36C prior to insertion into platform 38. In some implementations, connection between compression flange 26G and connector 36C is formed by adhesive, or spot welding, or other connection means.

In some implementations, compression flange 26G is provided as a separate part from connector 36C. As such, compression flange 26G is thereby substantially secured to connector 36C by pressure between the outer wall of connector 36C compressing compression flange 26G against the inner wall of platform 38 that is the result of forcefully securing connector 36C to platform 38.

As connector 36C is secured to platform 38, the surfaces of compression flange 26G are thereby substantially compressed between the outer wall of connector 36C and the inner wall of platform 38. The compression pressure is sufficient to bioseal the surfaces of compression flange 26G by substantially eliminating spaces within the connection area. A Morse taper configuration of connector 36C and platform 38 further facilitates the biosealing capability of the connection.

In some implementations, compression flange 26G is comprised of a very thin, flexible, layer of a high-durometer material, such as a metal or a hard plastic. In some implementations, compression flexible flange 26G is comprised of a thin layer of titanium, such as a titanium foil. In some implementations, the connection of compression flexible flange 26G to connector 36C and platform 38 can be considered to have achieved a cold weld connection. In some implementations, at least a portion of a metal foil-type compression flange 26G is connected to another material, such as a plastic, to enhance handling, durability, surface textures, and so on.

Compression flange 26G is selected to have a sufficiently large radius so as to be configurable for tucking into a small pouch formed under the perimeter of gingiva 34. Slots 32G that are interspersed over the upper and lower surfaces of compression flange 26G are biosealingly retentive of graft cover 46 shown having flowed partly into slots 32G. A membrane 44 biosealingly engages slots 32G between compression flange 26G and bone graft 28 in osseous void 30.

A variety of compression flange 26G configurations may be provided to facilitate convenient biosealing of bone graft 28 or gingiva 24 emergence profile formation, and so on. As such, users may elect to detach a first compression flange 26G after a given time, and then connect a second compression flange 26G. In some implementations, compression flange 26G has configurations that are different than the configuration shown in FIG. 7, including various thicknesses, different geometries, different surface features, and so on.

Graft cover 46 is shown biosealingly engaging slots 32G on the upper surface of compression flange 26G, and biosealingly contacting gingiva 34. After a short time, gingiva 34 is able to directly biosealingly engage compression flange 26G and slots 32G, whether or not graft cover 46 is present.

In some implementations, compression flange 26G is located at various elevations with respect to body 24G, including at bone-level, at tissue-level, and other preferred levels. Various vertical thicknesses of compression flange 26G may be provided to enhance ease of use, and biosealing properties.

In some implementations, body 24G is comprised of a hard, high-durometer material. In some implementations, a compression flange 26G is used with a hard body 24G for a given period during the healing of bone graft 28 and the osseointegration of implant 22.

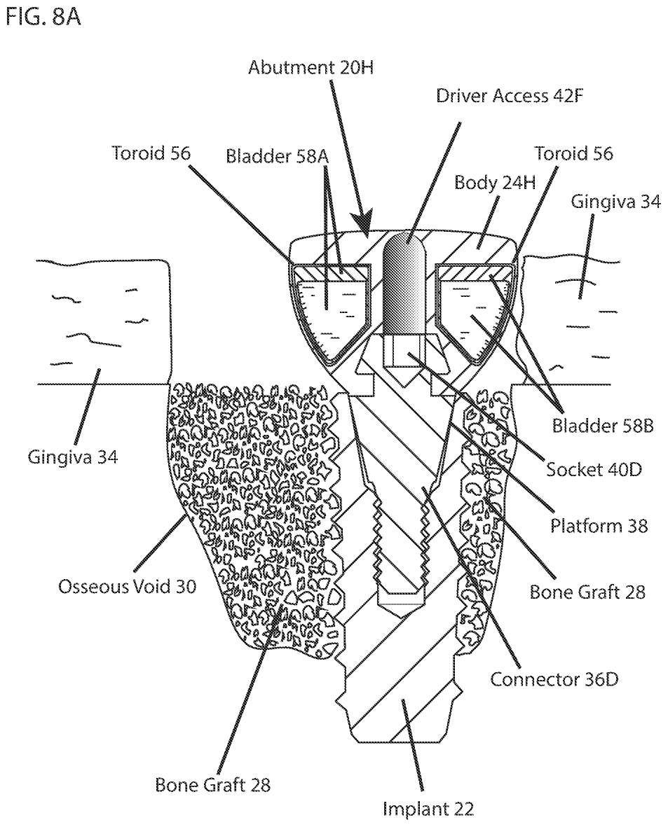

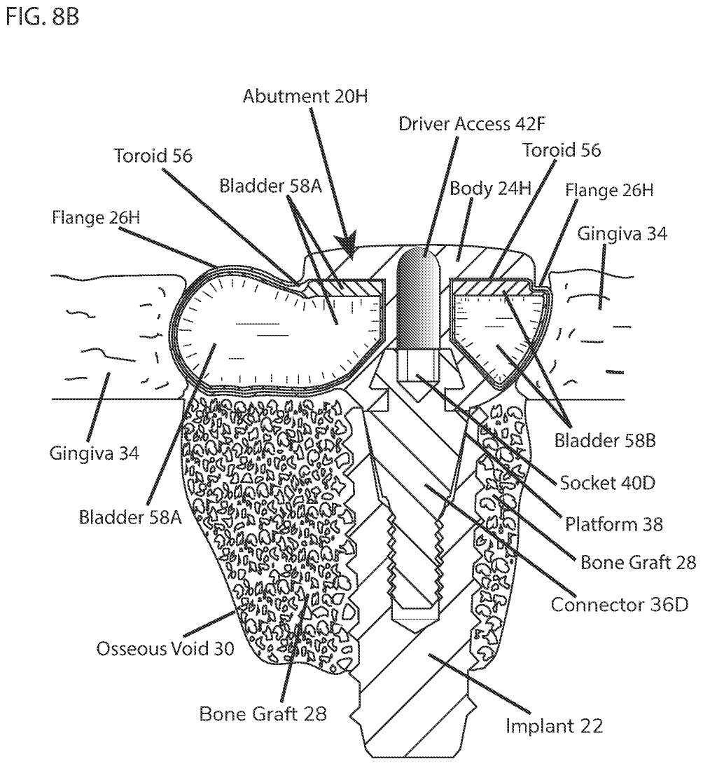

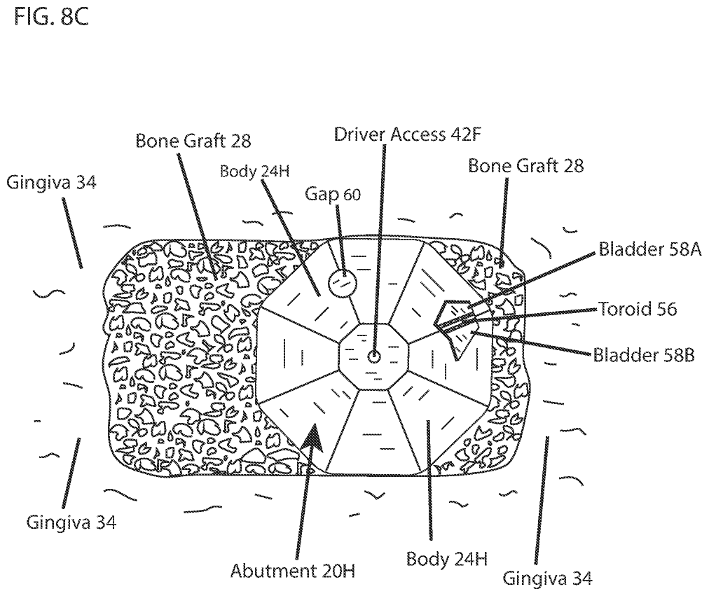

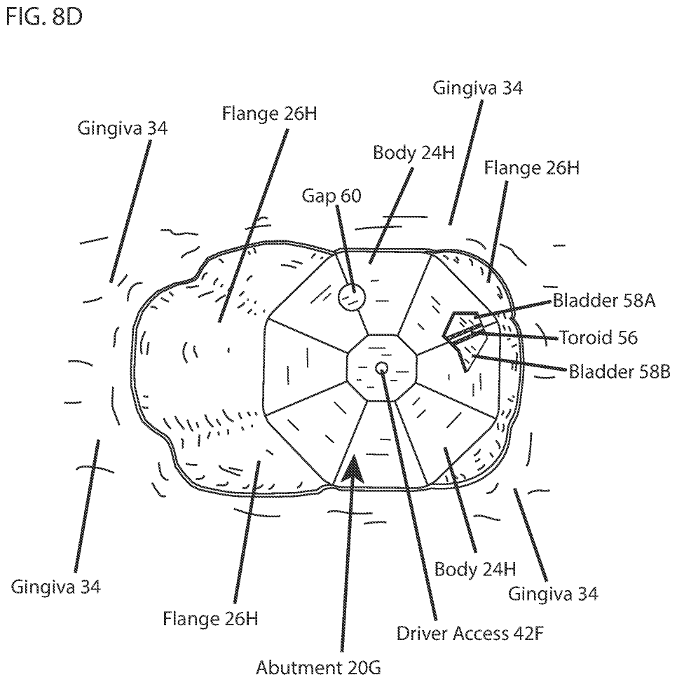

In accordance with an aspect and referring to FIG. 8A, there is provided an abutment 20H for securing to an implant 22. Connector 36D connects a soft body 24H to platform 38 of implant 22. Driver access 42F provides access for a driver tool to socket 40D. Bone graft 28 fills osseous void 30 up to the level of gingiva 34.

Toroid 56 contains a bladder 58A, bladder 58B, and other bladders 58 not shown. The external walls of toroid 56 are readily distendable laterally when bladders 58A/B are inflated. The upper portion of body 24H is thickened to resist distension, as is the internal wall between toroid 56 and driver access 42F. In some implementations, bladders 58 contain a unique dye color that may be aspirated into a syringe to confirm accurate needle location within a given bladder 58.

In accordance with an aspect and referring to FIG. 8B, there is provided an abutment 20H for securing to an implant 22. A connector 36D connects a soft body 24H to platform 38 of implant 22. A driver access 42F provides access for a driver tool to socket 40D. Bone graft 28 fills osseous void 30 up to the level of gingiva 34.

Toroid 56 contains a bladder 58A, bladder 58B, and other bladders 58 not shown. The walls of toroid 56 are shown distended laterally from the force of inflation of bladders 58A and 58B with an injected fluid or gas. The lateral distension of toroid 56 has formed an expandable flange 26H that biosealingly contains bone graft 28, and biosealingly contacts gingiva 34. The thickened upper top portion of body 24H resists distension, as does the internal wall between toroid 56 and driver access 42F.

In accordance with an aspect and referring to FIG. 8C, there is provided an abutment 20H for securing to an implant. A driver access 42F provides access for a driver tool to engage a drive socket inside driver access 42F. Bone graft 28 is surrounded by a gingiva 34 perimeter.

Body 24H is comprised of an elastomeric material. Body 24H contains toroid 56, shown in a cutaway. Toroid 56 contains bladders 58A and 58B, shown in the cutaway, and a multiplicity of other bladders 58. Markings on the upper exposed surface of soft body 24H identify the locations of the bladders 58 to facilitate accurate injection for the inflation of the bladders 58.

A gap 60 identifies an injection site where fluid or gas may be conveniently injected into toroid 56 in order to inflate and expand toroid 56 without inadvertently injecting into a bladder 58. When toroid 56 is pressurized by injection without pressurizing any bladders 58, then an expandable flange 26H forms, and wherein expandable flange 26H distends laterally symmetrically about the perimeter of body 24H. The gap 60 injection site comprises a substantial space between adjacent bladders 58 for convenient needle placement.

The gap 60 injection site is formed by an indention into the wall of at least one bladder 58, or by opposing indentions into the walls of two adjacent bladders 58. The dimensions of the gap 60 injection site is sufficient that positioning a needle within the gap 60 injection site is convenient. In some implementations, toroid 56 contains a unique dye color that may be aspirated into a syringe to confirm accurate needle location within toroid 56.

Typically, a user injects into gap 60 to pressurize toroid 56 until an expandable flange 26H distends laterally symmetrically to contact the closest portion of the gingiva 34 perimeter. Subsequent injections into multiple bladders 58 can cause sufficient lateral asymmetric distension of body 24H, such that expandable flange 26H distends sufficiently to biosealingly contact gingiva 34 along any remaining uncontacted portions of the gingiva 34 perimeter.

In accordance with an aspect and referring to FIG. 8D, there is provided an abutment 20H for securing to an implant. A driver access 42F provides access for a driver tool to engage a drive socket.

Body 24H is comprised of an elastomeric material. Body 24H contains toroid 56, shown in a cutaway. Toroid 56 contains bladders 58A and 58B, shown in the cutaway, and a multiplicity of other bladders 58. Markings on the upper exposed surface of soft body 24H identify the locations of the bladders 58 to facilitate accurate injection for the inflation of the bladders 58.

A gap 60 marking identifies the location of an injection site where fluid or gas was injected into the toroid 56 space in order to inflate toroid 56 without inadvertently injecting into a bladder 58. As toroid 56 is pressurized by injection, without pressurizing any bladders 58, an expandable flange 26H is formed, wherein expandable flange 26H distends laterally symmetrically about the perimeter of body 24H.

Subsequent injections into multiple bladders 58 cause sufficient lateral asymmetric distension of body 24H, such that expandable flange 26H distends sufficiently to biosealingly contact gingiva 34 along any remaining portions of the gingiva 34 perimeter.

After adequate stabilization of bone graft, a needle can be inserted into bladders 58, toroid 56, or both, to withdraw a portion of the fluid or gas therein. As fluid or gas is partly withdrawn, elasticity of the walls of toroid 56, bladder 58, or both, causes the configuration of expandable flange 26H to partly rebound in the direction of the original uninflated configuration. As such, body 24H can be reconfigured to facilitate the formation of an ideal gingiva 34 emergence profile.

### Method

In accordance with another aspect, there is provided a method for biosealing containment of a bone graft in an osseous void comprising the steps of: inserting an implant and bone graft into an osseous void, connecting a biosealed flexible flange to the implant, and configuring the flange to biosealingly contact the gingival perimeter.

In some implementations, the flexible flange extends laterally from a healing abutment body. In some implementations, a graft cover material is placed to further enhance biosealing contact with the gingival perimeter. In some implementations, the flange is configured to facilitate the formation of an ideal gingival emergence profile.

In accordance with another aspect, and referring to FIG. 1, a user places an implant 22 and bone graft 28 into osseous void 30. Gingiva 34 is lifted from contact with the bone around the perimeter of osseous void 30 to form a small pouch.

An abutment 20A is selected having a membrane 44 pre-connected to bone-level flange 26A. A driver tool is inserted into drive access 42A of abutment 20A to engage socket 40A. Abutment 20A is carried and rotationally inserted into implant 22 using the driver tool. The driver tool secures connector 36A to platform 38, and is removed.

After insertion, the bone-level flange 26A configuration is custom fitted by the user, such as by using scissors, or a rotary instrument. The perimeter edge of bone-level flange 26A is tucked into the gingiva 34 pouch, and over the perimeter bone about osseous void 30, thereby forming a biosealing contact with gingiva 34, and biosealingly containing bone graft 28. Membrane 44 engages slots 32A, and contacts the perimeter bone, further containing and biosealing bone graft 28.

In some implementations, graft cover 46 is placed over the top of bone-level flange 26A, and in contact with the perimeter of gingiva 34, further biosealingly containing bone graft 28. Graft cover 46 flows into and biosealingly engages slots 32A, further biosealingly containing bone graft 28.

During the initial minutes and hours, gingiva 34 increasingly engages bone-level flange 26A by microscopic sealing contact against the surface of bone-level flange 26A, and by protruding into and retentively engaging slots 32A. As such, gingiva 34 forms biosealing containment of bone graft 28 with bone-level flange 26A. The containing connection of gingiva 34 with bone-level flange 26A continues to increase over time, further enhancing the biosealing containment of bone graft 28. Gingiva 34 is able to directly biosealingly engage bone-level flange 26A and slots 32A, whether or not graft cover 46 is present.

After a healing period when bone graft 28 has stabilized, the user further reconfigures bone-level flange 26A, membrane 44, and graft cover 46, such that an ideal gingiva 34 emergence profile will form. After a further healing period when gingiva 34 has formed an ideal emergence profile, abutment 20A, membrane 44, and graft cover 46, are removed from implant 22 and bone graft 28. A prothesis is secured to platform 38, wherein the prosthesis has an ideal emergence profile that matches the formed gingival 34 emergence profile. The prosthesis also has a wide and strong subgingival connection to implant 22, which was facilitated by the ideal gingiva 34 emergence profile formed by abutment 20A.

In accordance with another aspect, and referring to FIG. 3, a user places an implant 22 and bone graft 28 into osseous void 30. A thin layer of graft cover 46 is placed directly over bone graft 28.

An abutment 20B is selected having midlevel flange 26B. A driver tool is inserted into drive access 42B of abutment 20B to engage socket 40A. Abutment 20B is carried and rotationally inserted into implant 22 using the driver tool. The driver tool secures connector 36A to platform 38, and is removed.

After insertion, the bone-level flange 26B configuration is custom fitted by the user, such as by using scissors, or a rotary instrument. The perimeter of midlevel flange 26B makes biosealing contact with gingiva 34, thereby biosealingly containing bone graft 28. During the initial minutes and hours, gingiva 34 increasingly engages mid-level flange 26B by microscopic sealing contact against the perimeter of mid-level flange 26A, and by engaging slots 32A.

Midlevel flange 26B rests on top of, and in contact with, graft cover 46. Graft cover 46 flows into and biosealingly engages slots 32B.

The user inserts additional graft cover 46 over the top of midlevel flange 26B, and in contact with the gingiva 34 perimeter, further biosealingly containing bone graft 28. Gingiva 34 is able to directly biosealingly engage midlevel flange 26B and slots 32B, whether or not graft cover 46 is present.

After a healing period when bone graft 28 has stabilized, the user further trims midlevel flange 26B, and graft cover 46 if present, such that gingiva 34 will heal to form an ideal gingival emergence profile. After a further healing period when gingiva 34 has formed an ideal emergence profile, abutment 20B and graft cover 46 are removed from implant 22 and bone graft 28. A prothesis is secured to platform 38, wherein the prosthesis has an ideal emergence profile that matches the formed gingival 34 emergence profile. The prosthesis also has a wide and strong subgingival connection to implant 22, which was facilitated by the ideal gingiva 34 emergence profile formed by abutment 20B.

In accordance with another aspect, and referring to FIG. 4, a user places an implant 22 and bone graft 28 into osseous void 30. Graft cover 46 is placed directly over bone graft 28, and fills the space between gingiva 34 and body 24C up to the level of the gingiva 34 outer surface.

An abutment 20C is selected having tissue-level flange 26C. A driver tool is inserted into drive access 42C to engage socket 40A. Abutment 20C is carried and inserted into implant 22 by the driver tool. The driver tool rotationally secures connector 36A to platform 38, and is removed.

After insertion, the tissue-level flange 26C configuration is custom fitted by the user, such as by using scissors or a rotary instrument. The perimeter portion of tissue-level flange 26C overlaps the perimeter edge of gingiva 34. The remaining portions of tissue level flange 26C rests on top of, and in contact with, graft cover 46. Graft cover 46 flows into and biosealingly engages slots 32B. Tissue-level flange 26C biosealingly contains bone graft 28 and graft cover 46. After a short time, gingiva 34 is able to directly biosealingly engage tissue-level flange 26C and slots 32C, whether or not graft cover 46 is present.

After a healing period when bone graft 28 has stabilized, the user reconfigures tissue-level flange 26C and graft cover 46 to facilitate the formation of an ideal gingiva 34 emergence profile. After a further healing period when gingiva 34 has formed an ideal emergence profile, abutment 20C and graft cover 46 are removed from implant 22 and bone graft 28.

A prothesis is secured to platform 38, wherein the prosthesis has an ideal emergence profile that matches the formed gingival 34 emergence profile. The prosthesis also has a wide and strong subgingival connection to implant 22, which was facilitated by the ideal gingiva 34 emergence profile formed by abutment 20C.

In accordance with another aspect, and referring to FIG. 5A, a user places an implant 22 and bone graft 28 into osseous void 30. A small pouch is formed under the perimeter of gingiva 34 with a surgical instrument. An abutment 20D is selected having full-thickness flange 26D. A membrane 44 is preconnected to full-thickness flange 26D by engaging slots 32D on the undersurface. A driver tool engages socket 40B of connector 36B, carries connector 36B to implant 22, rotationally secures connector 36B to platform 38, and is removed.

Body 24D with full-thickness flange 26D is hovered over connector 36B in order to estimate the preferred configuration for fitting into the space over bone graft 28 and against the perimeter of gingiva 34. Full-thickness flange 26D is trimmed to the preferred configuration, such as with scissors or a rotary instrument. Configuration of full-thickness flange 26D exposes a multiplicity of previously hidden slots 32D along the perimeter of full-thickness flange 26D.

During trimming of full-thickness flange 26D, membrane 44 is configured so that the perimeter of membrane 44 extends a couple millimeters outwardly beyond the perimeter of full-thickness flange 26D.

Once configured, body 24D with full-thickness flange 26D is positioned over connector 36B, such that the snap 48A opening is aligned with the top of connector 36B. Body 24D is pressed down toward connector 36B such that the opposing extension of snap 48A expands to matingly slide onto the top of connector 36B. When snap 48A is fully seated over connector 36B, then body 24D with full-thickness flange 26D are secured to connector 36D. Snap 48A facilitates rapid and convenient insertion or removal of body 24D and full-thickness flange 26D from over bone graft 28.

The perimeter of full-thickness flange 26D abuts against the perimeter edge of gingiva 34. Body 24D and full-thickness flange 26D form a biosealed connection to connector 36B, such that bacteria are blocked from intruding from the oral cavity downward through body 24D and between snap 48B and connector 36B.

The perimeter of gingiva 34 is lifted slightly to open the pouch created between gingiva 34 and bone 50. The extended perimeter of membrane 44 is tucked into the pouch formed under gingiva 34, and gingiva 34 is pressed down onto the upper surface of membrane 44, further containing and biosealing bone graft 28.

Graft cover 46 is shown in the perimeter area between gingiva 34 and full-thickness flange 26D. Graft cover 46 flows into and engages slots 32D that are exposed along the perimeter of full-thickness flange 26D to further facilitate biosealing bone graft 28. Graft cover 46 may comprise ambient blood. After a short time, gingiva 34 biosealingly engages the lateral surface of full-thickness flange 26D, and biosealingly engages slots 32D.

After a healing period when bone graft 28 has stabilized, the user has a first option to reconfigure full-thickness flange 26D to facilitate the formation of an ideal gingiva 34 emergence profile. The user also has a second option to select a body 24E with profile flange 26E preconfigured to facilitate an ideal gingiva 34 emergence profile, which are shown in FIG. 5B. Body 24E and profile flange 26E are also user-configurable as needed.

Body 24E is rotationally oriented over connector 36B until profile flange 26E extends asymmetrically away from connector 36B, and outwardly over a portion of stabilized bone graft 28. Body 24E is secured to connector 36B by pressing firmly over the snap 48B area until snap 48B flexes over the height of contour and fully seats into the undercut area of connector 36B. Body 24E and profile flange 26E are configured and oriented to facilitate formation of an ideal gingiva 34 emergence profile.

After a further healing period, an ideal gingiva 34 emergence profile is formed by the healing growth of gingiva 34 to intimately contact the lateral perimeter surfaces of body 24E and profile flange 26E, and engage slots 32E, as shown in FIG. 5B. As such, body 24E and profile flange 26E bioseal the spaces over bone graft 28.

After osseointegration of implant 22, abutment 20E is removed from implant 22 by first forcefully lifting body 24E away from connector 36B such that snap 48B expands over the height of contour of connector 36B, and is thereby released from the undercut. Second, connector 36B is rotationally removed from platform 38.

A prothesis is secured to platform 38, wherein the prosthesis has an ideal emergence profile that matches the formed gingival 34 emergence profile. The prosthesis also has a wide and strong subgingival connection to implant 22, which was facilitated by the ideal gingiva 34 emergence profile formed by abutment 20E.

In accordance with another aspect, and referring to FIG. 6, a user places an implant 22 and bone graft 28 into osseous void 30. A thin layer of graft cover 46 is placed directly over bone graft 28.

An abutment 20F is selected having a first detachable flange 26F that is biosealingly connected to body 24F by pellicle 54, and by elastomeric pressure retention within groove 52. A driver tool is inserted into drive access 42D of abutment 20F to engage socket 40A. Abutment 20F is carried and inserted into implant 22 by the driver tool. The driver tool rotationally secures connector 36A to platform 38, and is removed.

After insertion, detachable flange 26F is custom reconfigured by the user, such as by using scissors or a rotary instrument. The perimeter of detachable flange 26F biosealingly abuts against gingiva 34, thereby biosealingly containing bone graft 28. Detachable flange 26F covers and contains graft cover 46. Graft cover 46 flows into and engages slots 32F of detachable flange 26F, further biosealingly containing bone graft 28. The user inserts additional graft cover 46 over the top surface of detachable flange 26F with slots 32F, and in contact with the gingiva 34 perimeter, further biosealingly containing bone graft 28. After a short time, gingiva 34 directly biosealingly engages detachable flange 26F and slots 32F, whether or not graft cover 46 is present.

After a healing period when bone graft 28 has stabilized, detachable flange 26F is intentionally detached from abutment 20F, wherein biosealing pellicle 54 readily tears to release detachable flange 26F. Graft cover 46 is also removed.

A second detachable flange 26F is connected to body 24F, such as by retentively and elastomerically engaging an inner perimeter portion of second detachable flange 26F into groove 52. Second detachable flange 26F is configured to facilitate the formation of an ideal gingiva 34 emergence profile. Second detachable flange 26F typically has a thickness, vertical height, and overall configuration that facilitates the formation of an ideal emergence profile of gingiva 34. After a short time, gingiva 34 is able to directly biosealingly engage the second detachable flange 26F and slots 32F, whether or not graft cover 46 is present.

After a further healing period when gingiva 34 has formed an ideal emergence profile, abutment 20F and second detachable flange 26F are removed from over implant 22 and bone graft 28.

A prothesis is secured to platform 38, wherein the prosthesis has an ideal emergence profile that matches the formed gingival 34 emergence profile. The prosthesis also has a wide and strong subgingival connection to implant 22, which was facilitated by the ideal gingiva 34 emergence profile formed by abutment 20F.

In accordance with another aspect, and referring to FIG. 7, a user places an implant 22 and bone graft 28 into osseous void 30. Gingiva 34 is lifted from contact with bone 50 to form a small pouch.

A compression flange 26G having a preconnected membrane 44 is selected, and hovered over implant 22 to estimate the trimmed configuration. Compression flange 26G is custom trimmed to fit gingiva 34 perimeter, such as with scissors or a rotary tool. The central hole in compression flange 26G is centered over platform 38. Compression flange 26G is nestingly fitted into platform 38.

A driver tool is inserted into drive access 42E to engage socket 40C. Preassembled body 24G and connector 36C are carried and nested into the central hole area of compression flange 26G, and into platform 38, by the driver tool. The driver tool rotationally secures connector 36C to platform 38, and is removed.

Pressure on the surfaces of compression flange 26G, which are compressed by forceful contact between connector 36C and platform 38, create and comprise a substantial connection of connector 36C to compression flange 26G. As such, the connection between connector 36C and compression flange 26G forms a bioseal, even without the presence of ambient blood.

The perimeter of compression flange 26G is tucked into the gingiva 34 pouch, and over bone 50. Membrane 44 contacts bone 50, further containing and biosealing bone graft 28.

The user inserts graft cover 46 over the top of compression flange 26G, and in contact with the gingiva 34 perimeter, further biosealingly containing bone graft 28. Graft cover 46 flows into and biosealingly engages slots 32G, further biosealingly containing bone graft 28. Local ambient blood in contact with compression flange 26C further bioseals bone graft 28.

Within a short time, gingiva 34 increasingly biosealingly engages compression flange 26F by contact against the surface of compression flange 26F, and by protruding into and retentively engaging slots 32G. As such, compression flange 26F forms biosealing containment of bone graft 28. The containing connection of gingiva 34 with compression flange 26G continues to increase over time, further enhancing the biosealing containment of bone graft 28. After a short time, gingiva 34 is able to directly biosealingly engage the compression flange 26G and slots 32G, whether or not graft cover 46 is present.

After a healing period when bone graft 28 has stabilized, the user further trims compression flange 26G, membrane 44, and graft cover 46, to facilitate the formation of an ideal gingiva 34 emergence profile. After a further healing period when gingiva 34 has formed an ideal emergence profile, abutment 20G, compression flange 26G, membrane 44, and graft cover 46, are removed from implant 22 and bone graft 28.

A prothesis is secured to platform 38, wherein the prosthesis has an ideal emergence profile that matches the formed gingival 34 emergence profile. The prosthesis also has a wide and strong subgingival connection to implant 22, which was facilitated by the ideal gingiva 34 emergence profile formed by abutment 20G, compression flange 26G, and graft cover 46.

In accordance with another aspect, and referring to FIG. 8A-8D, an implant 22 and bone graft 28 are placed into osseous void 30. An abutment 20H is selected having a toroid 56, bladders 58A and 58B, and a multiplicity of other bladders 58. A driver tool is inserted into driver access 42F to engage socket 40D. Connector 36D is rotationally secured into platform 38, as shown in FIG. 8A.

A syringe having a needle is filled with sterile saline. The needle is inserted into gap 60 by inserting through the center of the gap 60 marking on the top surface of body 24H, as shown in FIG. 8C. A unique dye contained in toroid 56 is aspirated into the syringe, confirming to the user that the needle is positioned within the lumen of the gap 60 space between bladders 58.

Saline is injected into toroid 56 to pressurize and distend toroid 56. As toroid 56 is pressurized and distended, the thin, elastomeric, lateral walls of toroid 56 and body 24H symmetrically distend a distance away from the top of body 24H to form an expandable flange 26H. Toroid 56 is further pressurized and distended until a portion of expandable flange 26H biosealingly contacts the closest portion of the gingiva 34 perimeter. The needle is withdrawn from gap 60, and the thickened stopper-like top portion of body24H seals off the needle hole to prevent inadvertent leakage of saline from pressurized toroid 56.

The needle is centered over the markings on the top of body 24H that indicate the location of a bladder 58 below the surface. The needle is inserted through the top of body 24H and into a first bladder 58. A unique dye contained in the first bladder 58 is aspirated into the syringe, indicating to the user that the needle is positioned within the first bladder 58 lumen. Saline is injected into the first bladder 58 to pressurize and distend the first bladder 58. The thin elastomeric walls of the first bladder 58 distend laterally, and thereby asymmetrically and laterally distend the adjacent thin elastomeric lateral walls of toroid 56 a distance away from the top of body 24H to form an expandable flange 26H.

As saline is further injected into the first bladder 58, the adjacent lateral wall of toroid 56 is asymmetrically distended until expandable flange 26H biosealing contacts the adjacent portion of the gingiva 34 perimeter. The needle is withdrawn from the first bladder 58 and from the top of body 24H. The thickened stopper-like tops of the first bladder 58 and body 24H prevents the pressurized saline from inadvertently leaking out of the first bladder 58 and out of toroid 56, respectively.

The needle is similarly inserted into a second bladder 58, and saline is similarly injected to pressurize the second bladder 58. The lateral walls of the second bladder 58 and toroid 56 are similarly distended, thereby forming an expandable flange 26H that asymmetrically laterally distends until brought into biosealingly contact with the adjacent gingiva 34 perimeter, as shown in FIG. 8B. The needle is withdrawn from the top of the second bladder 58, and from the top of body 24H.

The needle injection process is repeated for other bladders 58 as needed to ensure that expandable flange 26H is asymmetrically extended sufficiently over bone graft 28 to biosealingly contact the entire surrounding gingiva 34 perimeter, as shown in FIG. 8D. Bone graft 28 is thereby biosealingly contained within osseous void 30. The bladders 58 tend to remain pressure inflated to the various degrees attained during the initial placement of abutment 20H, such that expandable flange 26H remains in biosealing contact with the entire gingiva 34 perimeter until stabilization of bone graft 28 has occurred.

After stabilization of bone graft 28, a needle is reinserted into a first bladder 58. A portion of the saline contained in the first bladder 58 is withdrawn to partly depressurize the first bladder 58. As the first bladder 58 is partly depressurized, the distended lateral walls of the first bladder 58 and toroid 56, and the related portion of expandable flange 26H, partly elastomerically retract toward the center of body 24H. Once the portion of expandable flange 26H has sufficiently retracted to a preferred location over bone graft 28, such as to facilitate formation of an ideal gingiva 34 emergence profile, then the needle is withdrawn from the first bladder 58 and body 24H.

A similar process of withdrawing saline and partly depressurizing a second bladder 58 is performed until the associated portion of expandable flange 26H has retracted to a unique preferred location over bone graft 28. The process of withdrawing saline and partly depressurizing other bladders 58 is repeated as needed until each associated portion of expandable flange 26H is reduced and retracted to a preferred degree of partial extension over bone graft 28. As such, the resulting overall perimeter of expandable flange 26H is substantially controllable to achieve a preferred circumferential configuration, such as a configuration that would facilitate the formation of an ideal gingiva 34 emergence profile.

A multiplicity of other methods are possible utilizing the flexible flanged abutments shown to bioseal bone graft 28 at initial placement, and to form an ideal gingiva 34 emergence profile.

1. A bone graft biosealing healing abutment having a biosealingly connected flexible flange for use in combination with an implant that is implanted into an osseous structure, wherein said osseous structure has a bone graft extending a given distance laterally from said implant, and wherein a gingival perimeter surrounds said bone graft, wherein said flange is configurable to extend laterally said distance from said implant, and wherein said flange biosealingly contacts said gingival perimeter to biosealingly contain said bone graft.

2. The abutment of claim 1, wherein said flange is comprised of a flexible material that is integral and continuous with a low durometer flexible material that comprises a soft body portion of said abutment.

3. The abutment of claim 1, wherein said flange is comprised of a flexible configuration of a high durometer material that is continuous with a hard, high durometer portion of said abutment.

4. The abutment of claim 1, wherein said flange is detachably connectable to said abutment.

5. A method for biosealingly containing a bone graft in an osseous void comprising the steps of: inserting an implant and bone graft into an osseous void, wherein said osseous void has a gingival perimeter, connecting a biosealing flexible flange to said implant such that said flange biosealingly contacts said gingival perimeter, and thereby biosealingly contains said bone graft.

6. The method of claim 5, wherein the flange is configured to facilitate the formation of an ideal gingival emergence profile after a bone graft stabilization time period.

7. A method for securing a prosthesis to an implant comprising the steps of: inserting an implant and a bone graft into an osseous void, wherein said osseous void has a gingival perimeter, securing a healing abutment to said implant, wherein said abutment has a flexible flange extending laterally therefrom into biosealingly contact with said gingival perimeter, such that said bone graft is biosealingly contained, waiting a healing time period, removing said abutment from said implant, and securing said prosthesis to said implant.

8. The method of claim 7, wherein the flange is configured to facilitate the formation of an ideal gingival emergence profile after a bone graft stabilization time period.
